(19) United States
(12) Patent Application Publication
(10) Pub. No.: US 2021/0198613 A1
(43) Pub. Date: Jul. 1, 2021

(54) MICROELECTRODE ARRAY AND USES THEREOF

(71) Applicants: AXOSIM, INC., New Orleans, LA (US); UNIVERSITY OF CENTRAL FLORIDA RESEARCH FOUNDATION, INC., Orlando, FL (US)

(72) Inventors: J. Lowry Curley, New Orleans, LA (US); Michael James Moore, New Orelans, LA (US); Corey Michael Rountree, New Orleans, LA (US); Hieu Trung Nguyen, New Orleans, LA (US); Swaminathan Rajaraman, Orlando, FL (US); Avra Kundu, Orlando, FL (US)

(21) Appl. No.: 17/193,589

(22) Filed: Mar. 5, 2021

Related U.S. Application Data
(63) Continuation of application No. PCT/US2019/049802, filed on Sep. 5, 2019.
(60) Provisional application No. 62/727,494, filed on Sep. 5, 2018.

(51) Int. Cl. C12M 1/34; G01N 33/483; G01N 33/50

(52) U.S. Cl. CPC C12M 41/46; G01N 33/5058; G01N 33/5014; G01N 33/4836

(57) ABSTRACT

The present invention is directed to a microelectrode array for use in microengineered physiological systems and methods of using the same.

### CROSS REFERENCE TO RELATED APPLICATIONS

This application claims the benefit of U.S. Provisional Application No. 62/727,494 filed Sep. 5, 2018, the entire contents of which are incorporated herein by reference.

### STATEMENT REGARDING FEDERALLY SPONSORED RESEARCH OR DEVELOPMENT

This invention was made with government support under grant R43ES029886-01 awarded by the National Institutes of Health. The Government has certain rights in the invention.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The present invention is directed to a microelectrode array for use in microengineered physiologic systems and methods of using the same.

### BACKGROUND OF THE INVENTION

The average new drug requires nearly $2.6 billion and up to 15 years to obtain market approval, as well as an additional $312 million for post-approval research and development to maintain approval. Unfortunately, there is a poor track record of drug development in conventional preclinical models leading to successful clinical therapeutics. For neurological applications in particular, it is estimated that as high as 92% of neurological drugs that enter Phase I clinical trials will never be marketed to consumers due either to unacceptable toxicity or lack of efficacy in humans. Clearly, current preclinical models including both animal and in vitro models have very limited predictivity when it comes to the translation of preclinical success to clinical trials. Animal models may provide relevant in vivo information, but they are time-consuming and labor intensive (low throughput), while on the other hand, higher throughput in vitro systems are typically restricted to basic neural cultures consisting of randomly growing dissociated cells in two dimensions and incapable of providing relevant in vivo information. Thus, higher throughput systems capable of providing relevant in vivo metrics are highly desired.

### SUMMARY OF THE INVENTION

One aspect of the invention is directed to a three-dimensional microelectrode array. In one embodiment, the microelectrode array comprises a chip that further comprises at least one two-dimensional electrode, at least one three-dimensional electrode, or a combination thereof. In one embodiment, the microelectrode array comprises at least one two-dimensional electrode. In an embodiment, the microelectrode array comprises at least one three-dimensional electrode. The microelectrode array can be configured to provide real-time, reliable detection of one or more bioelectrical signals in a microengineered physiological system. In certain embodiments, the one or more bioelectrical signals comprise single action potentials, compound action potentials, high frequency waves, low frequency waves, or a combination thereof. In one embodiment, the bioelectrical signal comprises a compound action potential. In embodiments, the microengineered physiological system comprises a tissue explant, a suspension of cells, or a combination thereof. The microengineered physiological system can comprise any of various neural cell types aggregated into a spheroid mass. The microengineered physiological system can comprise neural cells cultured on a micropatterned platform. In embodiments, the microengineered physiological system comprises tissue explants seeded on a micropatterned platform. The micropatterned platform can be configured to permit the formation of a neural architecture. In embodiments, the microelectrode array comprises an area with a configuration that is complementary to that of the neural architecture.

In certain embodiments, the neural architecture comprises an axonal growth region, a ganglion region, a dendritic region, a synaptic region, a spheroid region, or a combination thereof. The microelectrode array can comprise a first plurality of electrodes positioned in the ganglion region or spheroid region and a second plurality of electrodes positioned at defined intervals down the axonal growth region, dendritic region, synaptic region, or a combination thereof. The microelectrode array can include any of various electrodes known to those of skill in the art. In embodiments, the first plurality of electrodes, the second plurality of electrodes, or both comprise recording electrodes, stimulation electrodes, or a combination thereof. In certain embodiments, the first plurality of electrodes, the second plurality of electrodes, or both comprise at least one microneedle electrode, at least one planar electrode, or a combination thereof. In one embodiment, the first plurality of electrodes, the second plurality of electrodes, or both comprise at least one microneedle electrode. In an embodiment, the first plurality of electrodes, the second plurality of electrodes, or both comprise at least one planar electrode.

In embodiments, the electrodes can comprise any size appropriate for recording or stimulating microengineered physiological systems. In embodiments comprising at least one planar electrode, the at least one planar electrode can comprise a length of up to about 100 μm. The planar electrode can comprise a length of up to about 5 mm. The planar electrode can comprise a length of up to about 5 mm, 4 mm, 3 mm, 2 mm, or 1 mm. In embodiments, the length of the planar electrode is less than about 1 mm. The planar electrode can comprise a length of less than 500 μm. In embodiments, the planar electrode comprises a length as short as about 10 μm. The planar electrode can comprise a length of up to about 100 μm. In one embodiment the at least one planar electrode comprises a length of between 20 μm to about 80 μm, inclusive. The planar electrode can comprise a length of about 50 μm. In some embodiments, the planar electrode comprises a length of about 50 μm, about 40 μm, about 30 μm, about 20 μm, or about 10 μm In embodiments, at least one of the electrodes comprises a substantially square planar electrode.

At least one of the electrodes can comprise a three-dimensional electrode. In one embodiment, the at least one three-dimensional electrode comprises a base with a diameter of up to about 1000 μm. The three-dimensional electrode can comprise a base with a diameter of up to about 500 μm. In embodiments, the base of the three-dimensional electrode comprises a diameter of between about 75 μm to about 350 μm, inclusive. The base of the three-dimensional electrode can comprise a diameter of between about 100 μm to about 300 μm. The three-dimensional electrode can comprise a base with a diameter of about 500 μm, 400 μm, 300 μm, 200 μm, or 100 μm. In certain embodiments, the base comprises a diameter of about 250 μm. The diameter of the base can be less than 100 μm. In embodiments, the diameter of the base is about 100 μm, about 90 μm, about 80 μm, about 70 μm, about 60 μm, about 50 μm, about 40 μm, about 30 μm, about 20 μm, or about 10 μm. In embodiments, the height of the three-dimensional electrode can be between 1 μm to about 1000 μm. The three-dimensional electrode can comprise a height of up to about 1000 μm. The three-dimensional electrode can comprise a height of between about 30 μm to about 1000 μm. The three-dimensional electrode can comprise a height of up to about 800 μm. In embodiments, the height of the three-dimensional electrode is between about 100 μm to about 500 μm, inclusive. The height of the three-dimensional electrode can be between about 250 μm to about 450 μm, inclusive. In certain embodiments, the height of the three-dimensional electrode can be between about 350 μm and 450 μm, inclusive. In embodiments, the three-dimensional electrode comprises a height of up to about 150 μm. In certain embodiments, the three-dimensional electrode comprise a height of between about 50 μm to about 150 μm. The three-dimensional electrode can comprise a height of about 800, 700 μm, 600 μm, 500 μm, 400 μm, 300 μm, 200 μm, or 100 μm. In certain embodiments, the height of the three-dimensional electrode is about 450 μm. The height of the three-dimensional electrode can be less than 100 μm. In embodiments, the height of the three-dimensional electrode is about 100 μm, about 90 μm, about 80 μm, about 70 μm, about 60 μm, about 50 μm, about 40 μm, about 30 μm, about 20 μm, or about 10 μm. In certain embodiments, the at least one three-dimensional electrode comprises a tip with a radius of curvature (ROC) that is between 1 μm and 1 mm, inclusive. In certain embodiments, the ROC is less than about 50 μm. The ROC can be between about 5 μm to about 30 μm. In one embodiment, the ROC is about 15 μm

In embodiments, the microelectrode array comprises at least one electrode with a diameter of up to about 5 mm. The diameter of at least one electrode can be up to about 5 mm, 4 mm, 3 mm, 2 mm, or 1 mm. In embodiments, the diameter of at least one electrode is less than about 1 mm. In embodiments, at least one electrode comprises a diameter of up to about 500 μm. At least one electrode can comprise a diameter of less than 500 μm. The microelectrode array comprises at least one electrode with a diameter of up to about 400 μm. In embodiments, the diameter of at least one electrode is between about 75 μm to about 350 μm, inclusive. The diameter of at least one electrode is between about 100 μm to about 300 μm. The microelectrode array comprises at least one electrode with a diameter of about 500 μm, 400 μm, 300 μm, 200 μm, or 100 μm. In certain embodiments, the at least one electrode comprises a diameter of about 250 μm. The diameter of at least one electrode can be 50 μm or less. The diameter of at least one electrode can be about 30 μm or less. In certain embodiments, the microelectrode array comprises at least one electrode with a diameter of about 30-50 μm.

In embodiments, the defined intervals of the second plurality of electrodes down the axonal growth region comprise up to about 5 mm intervals. In embodiments, the defined intervals are at least 10 μm. In certain embodiments, the defined intervals comprise a distance of between about 10 μm to about 5 mm. The defined intervals can be between about 100 μm to about 1 mm. In embodiments, the defined intervals are about 1 mm.

The microelectrode array can comprise up to about 300 electrodes. In embodiments, the microelectrode array comprises up to about 200 electrodes. The microelectrode array can comprise up to about 100 electrodes. In certain embodiments, the microelectrode array comprises about 300, about 250, about 200, about 150, about 100, or about 50 electrodes. In certain embodiments, the microelectrode array comprises up to about seventy electrodes. In embodiments, the first plurality of electrodes and the second plurality of electrodes comprise up to a total of sixty-four electrodes when combined. In some embodiments, the first plurality of electrodes or the second plurality of electrodes comprises up to about sixty-four electrodes. The first plurality of electrodes or the second plurality of electrodes can comprise between about ten and about sixty-four electrodes. The first plurality of electrodes or the second plurality of electrodes can comprise between about twenty and about sixty electrodes. In certain embodiments, the first plurality of electrodes or the second plurality of electrodes comprises twenty, thirty, forty, fifty, or sixty electrodes. In embodiments, the first plurality of electrodes or the second plurality of electrodes comprise less than about twenty electrodes. In certain embodiments, the first plurality of electrodes comprises up to ten electrodes, the second plurality of electrodes comprises up to ten electrodes, or a combination thereof. The first plurality of electrodes, the second plurality of electrodes, or a both the first and second plurality of electrodes can comprise one, two, three, four, five, six, seven, eight, nine, ten, eleven, twelve, thirteen, fourteen, fifteen, sixteen, seventeen, eighteen, nineteen, or twenty electrodes. In one embodiment, the first plurality of electrodes comprises three electrodes and the second plurality of electrodes comprises seven electrodes. In an alternate embodiment, the first plurality of electrodes comprises six electrodes and the second plurality of electrodes can comprise nine electrodes. In embodiments, the microelectrode array is configured to accommodate at least 16 microneedle-type electrodes, at least 16 planar electrodes, or a combination thereof within the area that is complementary to that of the neural architecture.

The microelectrode array can be configured to detect bioelectric signals of at least about 10 μV. In embodiments, the microelectrode array is configured to detect bioelectric signals of between about 10 μV to about 100 μV, inclusive. The microelectrode array can be configured to detect bioelectric signals of about 10 μV, 20 μV, 30 μV, 40 μV, 50 μV, 60 μV, 70 μV, 80 μV, 90 μV, or 100 μV. The microelectrode array can be configured to detect bioelectric signals of at least about 40 μV.

In embodiments, the microelectrode array can be configured to detect bioelectric signals in a microengineered physiological system for an extended period of time. In embodiments, the microelectrode array is configured to detect bioelectric signals in a microengineered physiological system for at up to one year. The microelectrode array can be configured to detect bioelectric signals in a microengineered physiological system for up to about twelve months, eleven months, ten months, nine months, eight months, seven months, six months, five months, four months, three months, two months, or one month. In certain embodiments, the microelectrode array can be configured to detect bioelectric signals for up to 20 weeks. The microelectrode array can be configured to detect bioelectric signals for up to 10 weeks. The microelectrode array can be configured to detect bioelectric signals in a microengineered physiological system for at least one week, two weeks, three weeks, four weeks, five weeks, six weeks, seven weeks, eight weeks, nine weeks, or ten weeks. In embodiments, the microelectrode array is configured to detect bioelectric signals for at least eight weeks. The microelectrode array can be configured to detect bioelectric signals in a microengineered physiological system for between about four weeks to about eight weeks.

In one embodiment, the microelectrode array comprises a biocompatible conductive ink, a biocompatible conductive paste, a biocompatible conductive composite, or a combination thereof. The microelectrode array can comprise one or more vias.

In embodiments, the microelectrode array comprises an insulation layer. The insulation layer can comprise a material that is biocompatible. In one embodiment, the insulation layer is capable of being conformally coated at room temperature. In certain embodiments, the insulation layer comprises parylene, poly-di-methyl-siloxane (PDMS), SU-8, silicon dioxide, polyimide, polyurethane, poly lactic acid, poly glycolic acid, poly lactic glycolic acid, poly vinyl alcohol, polystyrene, poly ethylene glycol, poly ethylene terephthalate, poly ethylene terephthalate glycol, poly ethylene naphthalate, or a combination thereof.

The microelectrode array can further comprise volumetric stimulators configured to stimulate the microengineered physiological system.

In various exemplary embodiments, the microelectrode array is comprised of a biocompatible material. The microelectrode array can be configured to maintain viability of neuronal cells.

In embodiments, the microengineered physiological system comprises at least one cell with structural characteristics of cells within the central nervous system, the peripheral nervous system, or a combination thereof. In certain embodiments, the microengineered physiological system comprises at least one neuronal cell with structural characteristics of cells within a neural network disposed within a brain, a spinal cord, or a combination thereof. The microengineered physiological system can comprise at least one neuronal cell with a structure analogous to peripheral nerve anatomy. In certain embodiments, the microengineered physiological system comprises one or more synapses. In embodiments, the microengineered physiological system comprises at least one neuroendocrine synapse, at least one neuromuscular synapse, or a combination thereof.

In certain embodiments, the three-dimensional electrodes comprise microneedle-type electrodes.

In embodiments, the microelectrode array chip is configured to interface with standard commercial multichannel systems including standard commercial multichannel recording amplifiers. In embodiments, exemplary commercial systems and recording amplifiers include MCS, Axion, Plexon, Intan, NeuroNexus, and other known systems and amplifiers. The microelectrode array can be configured to measure an action potential for an inference of conduction velocity, amplitude, integral, excitability after compound administration, threshold, sensitivity, CAP time width, CAP waveform shape, or a combination thereof.

In certain embodiments, the microelectrode array comprises a conductive trace layer. In embodiments, the conductive trace layer comprises an electrically conductive material. The electrically conductive material can comprise titanium, titanium nitride, iridium oxide, platinum, gold, aluminum, stainless steel, indium tin oxide, or a combination thereof. In certain embodiments, the conductive material comprises a conductive polymer. Exemplary conductive polymers include polyethylenedioxythiophene (PEDOT), polypyrrole, polyaniline, or a combination thereof. In embodiments, titanium/aluminum trace layer, a polyethylene terephthalate insulation layer, micro-towers, or a combination thereof. In embodiments, the micro-towers are coated with micro-porous platinum, nano-porous platinum, nano-gold, or a combination thereof. The micro-towers can be insulated or non-insulated. In embodiments, the microelectrode array comprises a titanium/gold metal trace. The microelectrode array can comprise a titanium/aluminum trace layer.

Another aspect of the invention is directed to a system for reproducibly detecting compound action potentials in microengineered physiological system. In embodiments, the system comprises any of the various microelectrode arrays mentioned herein. The system can comprise a microphysiological system that further comprises one or more neural cells. In certain embodiments, the microengineered physiological system is grown on the microelectrode array. In embodiments, the microengineered physiological system is transferred to the microelectrode array. In embodiments, the one or more neural cells comprise peripheral nervous system neurons, central nervous system neurons, Schwann cells, oligodendrocytes, microglial cells, glial cells, or a combination thereof. In some embodiments, the one or more neuronal cells comprise sensory neurons, interneurons, or motor neurons. The peripheral nervous system neurons can comprise at least one dorsal root ganglion neuron.

One aspect of the invention is directed to a method of predicting the type and severity of a neural pathology. In embodiments, the method comprises growing neural tissue on any of the various microelectrode array systems disclosed herein, adding neural tissue to a microelectrode array system disclosed herein, adding a microelectrode array system to the neural tissue, or a combination thereof. The neural tissue can comprise an axonal growth region and a ganglion region. Electrophysiological testing can be performed to determine the nerve conduction velocity of the neural tissue. In certain embodiments, electrophysiological testing comprises stimulating at least one location along the axonal growth region, the ganglion region, or a combination thereof and recording from at least one location along the axonal growth region, the ganglion region, or a combination thereof. In embodiments, electrophysiological testing comprises electrically stimulating at least one location along the axonal growth region and recording from at least one location within the ganglion region. Alternate embodiments comprise electrically stimulating the ganglion region and recording from at least one location along the axonal growth region. In certain embodiments, reduced nerve conduction indicates a neural pathology. The method can further comprise histological analysis of the neural tissue. In embodiments, histological analysis comprises an assessment of axon diameter, axon density, myelination, cell morphology, cell type, nerve structure, or a combination thereof. In certain embodiments, electrophysiological testing can comprises stimulating a plurality of locations along the axonal growth region, the ganglion region, or a combination thereof and recording a resultant electrical response from the ganglion region, the axonal growth region, or a combination thereof. In embodiments, data obtained from histological analysis is correlated with data obtained from electrophysiological testing. Certain inferences of neural pathology can be drawn based on the correlation between the histological data and the electrophysiological data. Certain embodiments further comprise comparing nerve conduction velocity obtained from sample neural tissue to that of neural tissue that is known to be healthy neural tissue, wherein reduced nerve conduction in the sample neural tissue as compared to the healthy neural tissue indicates a neural pathology. In embodiments, relative changes in morphology, phenotype, genotype, structure, electrophysiology, or a combination thereof can be compared between sample neural tissue to that of healthy neural tissue or between sample neural tissue and neural tissue that has been subjected to at least one agent. In certain embodiments, the electrophysiological testing is performed over a multi-week period to chronically measure neurodegeneration.

Another aspect of the present invention is directed to a method of assessing a response from neural tissue. In embodiments, the method comprises growing neural tissue on any of the various microelectrode arrays disclosed herein, adding neural tissue to a microelectrode array system disclosed herein, adding a microelectrode array system to the neural tissue, or a combination thereof. The method can further comprise introducing one or more stimuli to the neural tissue; and measuring one or more responses from the neural tissue to the one or more stimuli. In embodiments, the one or more responses comprise compound action potential amplitude, conduction velocity, waveform shape, histomorphological parameters, or combination thereof. In embodiments, introducing the one or more stimuli comprises contacting the neural tissue with at least one pharmacologically active compound, electrical stimulus, chemical stimulus, optical stimuli, physical stimuli, or a combination thereof. In embodiments, optical stimuli includes engineered optical sensitivity through optogenetics or naturally expressed optical sensitivity through stimulation of photoreceptive neurons. Physical stimuli can include mechanical stimulation of neurons. In embodiments, mechanical stimulation can be achieved through activation of mechanosensitive channels such as, but not limited to, transient receptor potential vanilloid (TRPV) channel groups.

Yet another aspect of the present invention is direct to a method of evaluating the toxicity of an agent. In embodiments, the method comprises growing neural tissue on any of the various microelectrode arrays disclosed herein, adding neural tissue to a microelectrode array system disclosed herein, adding a microelectrode array system to the neural tissue, or a combination thereof. The method can further comprise exposing at least one agent to the neural tissue; measuring or observing changes in compound action potential amplitude, conduction velocity, waveform shape, histomorphological parameters, or combination thereof and correlating any measured or observed changes of the neural tissue with the toxicity of the agent, such that, if the measured or observed changes are indicative of decreased cell viability, the agent is characterized as toxic and, if the measured or observed changes are indicative of unchanged or increased cell viability, the agent is characterized as non-toxic.

One aspect of the present invention is directed to a method of measuring myelination or demyelination of one or more axons of one or a plurality of neuronal cells. In embodiments, the method comprises growing neural tissue on any one of the various microelectrode array embodiments disclosed herein under conditions sufficient to grow at least one axon, adding neural tissue to a microelectrode array system disclosed herein, adding a microelectrode array system to the neural tissue, or a combination thereof. The method can further comprise inducing a compound action potential in such neural tissue; measuring the compound action potential; and quantifying the levels of myelination of such neural tissue based on the compound action potential.

In another aspect, the present invention is directed to a method of fabricating a three-dimensional microelectrode array. In embodiments the method comprises processing a chip to accommodate a plurality of electrodes, a plurality of vias, or a combination thereof. The method can further comprise metallization of the plurality of electrodes using a shadow mask. In embodiments, the method includes screen printing of conductive inks. The method can further comprise curing the conductive ink in an oven. In certain embodiments, the method includes a step of depositing insulation onto the conductive ink and metalized electrodes. Insulation can also be deposited over the entirety of the processing chip. The method can include the step of defining the recording sites of the plurality of electrodes. In embodiments, a printed circuit board is combined with the chip. In certain embodiments, method further comprises fabricating conductive vias for top to bottom signal transduction.

Other objects and advantages of this invention will become readily apparent from the ensuing description.

### BRIEF DESCRIPTION OF THE FIGURES

FIG. 1 shows a peripheral Nerve-On-A-Chip® (AxoSim Technologies, LLC, New Orleans, La.) under one embodiment. A) Fluorescence image of construct with DAPI (blue) stained nuclei and β3-Tubulin (green) neurites. B) Brightfield image indicating recording and stimulation electrode placement. C) Confocal image stack of 3D neurite growth, with depth color-map. D) 3D orthographic view of confocal image stacks showing MBP-stained myelinated fibers near distal end of construct. E) TEM cross-section of indicating myelinated and bare axons as well as Schwann cells; inset=close-up of spiral, compact myelin structure. F) Representative images of healthy myelinated axon (upper left) as well as Fsk-induced dysmyelination (red arrows). G) Example traces of CAPs before and after Fsk administration (overlay of 10 consecutive recordings with average trace shown in red). H) Mean CAP amplitude and conduction velocity for control myelinated (M+), dexamethasone only (ODex), forskolin treated (Fsk) and forskolin with dexamethasone (DexM) coadministration; n=6, *p<0.05, ***p<0.001, ****p<0.0001.

FIG. 2 provides “Nerve-On-A-Chip®” 3D MEA device under one embodiment. Schematic (left) depicts fabrication process (left). Optical and SEM images of 3D electrodes (middle) designed to match engineered nerve tissue architecture. Full spectrum average impedance of the 3D MEA demonstrating reduced impedance post electroless platinum plating (N=3; top right); 10× increased charge carrying capacity of the 3D electrodes with micro-porous platinum (N=3; bottom right).

FIG. 3 depicts a schematic representation of an experimental setup under one embodiment with 3 electrodes stimulating different locations along the axon growth region and a recording electrode shown within the ganglion region.

FIG. 4 depicts an experimental design under one embodiment. In this embodiment, baseline physiological recordings are taken after growth and myelination in culture. Experiments include an acute (48 hr) application of each drug followed by an immediate or delayed (7 days) assessment by physiological recording (Rec) and imaging (CFM and TEM). The control group consists of vehicle administration, without drugs.

FIG. 5 shows a schematic of mask used for a custom solid-substrate MEA design under one embodiment. The boxed region is shown in greater detail to the right. Nine 50×50 μm electrodes are shown positioned in the region of the cell spheroids for recording responses, while six 100×500 μm electrodes are shown in 1-mm intervals down the length of the channel (only three are visible in close-up on the right).

FIG. 6 shows an exemplary process flow for fabrication of 3D MEAs: (a) 3D printing of base structure; (b) metallization through a micromilled stencil mask; (c) application of biocompatible laminate “gross” insulation layer and (d) assembling a 3D printed culture well onto the fabricated device. The close up of one of the recording/stimulating patches containing ten 3D electrodes is given for each process step; (e) Close up view of one of the patches after electroless plating of platinum; (0 Exploded view of the device showing the deposition of the “fine” SiO2 insulation layer after the metallization step. (g) Singular 3D microtower after deposition of SiO2; (h) Singular 3D microtower after laser micromachining of SiO2 insulation thereby exposing the metal underneath; (i) Singular 3D microtower with smaller microelectrodes after electroless plating of platinum.

FIG. 7 provides SEM images: (a) One of the recording/stimulating patches containing ten 3D microtowers; (b) Three microtowers in the circular region of the patch showing inherent striations after 3D printing due to layer by layer fabrication of SLA printing; (c) Smoothening of the microtower surface after acetone vapor polishing of the microtowers leading to a reduction in striations; (d) Close up of the tip of a singular 3D microtower depicting a radius of curvature of ˜15 μm. process.

FIG. 8 shows photomicrographs of the fabricated device under one embodiment: (a) Metallized device with (b) close up of the metallized 3D microtowers; (c) Application of the biocompatible laminate “gross” insulation layer indicated by a dotted circle and (d) assembled 3D MEA device in a 49 mm×49 mm×1 mm form factor for compatibility with amplifier setup.

FIG. 9 provides full spectrum (a) impedance and (b) phase characteristics of the 3D microtower MEAs. The line indicates the electrophysiologically significant 1 kHz values on the right hand side of the graph; (c) Optical micrographs of a Single 3D microtower MEA before (c) and after (d) electroless plating of micro-porous platinum. It is clear from the micrographs that micro-porous platinum has been deposited at the tips of the microtower.

FIGS. 10 (a) and (b) show the scan rate variation of cyclic voltammetry of the 3D microtower MEAs under one embodiment (a) before and (b) after electroless plating of platinum; (c) Extracted current vs. scan rate from (a) and (b) for estimation of the double layer capacitance values before and after micro-porous platinum plating; (d) Full spectrum impedance and phase response of the 30 μm×30 μm “fine” microelectrodes atop the 3D microtower before and after electroless plating of micro-porous platinum.

FIG. 11 provides (a) close-up photomicrograph of the tip of 3D microtower under one embodiment after SiO2 deposition depicting the purple hue of the SiO2 layer; (b) Distinctive micro-porous platinum on the “fine” microelectrodes after electroless plating of platinum subsequent to the laser micromachining of SiO2; (c) SEM image of the “fine” laser ablated, micro-porous platinum plated SiO2 electrode; (d) EDS analysis of the “fine” microelectrode after electroless plating of platinum on the islands of the microporous material formed.

FIG. 12 shows (a) DRGs on 3D Microtowers (marked in blue) of the MEA under one embodiment with (b) a close-up view of the Matrigel® Matrix keyhole (marked in blue). The outline of the PEG layer is marked in red. (c) Fluorescence microscopy of DRGs on 3D Microtowers (marked in yellow circles) of the MEA in the circular region of the Nerve-On-A-Chip®. (d) Stitched composite image depicting DRG placed onto the MEA (1), using Matrigel. DRG stained with calcein AM staining (green) and Propidium iodide staining (red) taken at 4× using and inverted Microscope. (2) Neural cells wrapped around 3D microtowers, determining cell biocompatibility. (e) Close up of the circular region of the Nerve-On-A-Chip® for the control sample.

FIG. 13 shows data from Nerve-On-A-Chip® biocompatibility obtained by measuring neural cell viability after 10 days' culture on an MEA under one embodiment. (a) The bar graphs compare the control (neural cell viability on tissue culture plastic) versus cells grown on insulated devices and plain resin. Error bars indicate SD and *** indicate significance of p<0.0001 for ANOVA. (b) FTIR analysis of the 3D printed clear resin with (c) exploded plot of the fingerprinting region. (d) Water sorption characteristics of the fabricated 3D MEAs and (e) SEM image of a 3D printed high density 3D MEA (base diameter ˜100 μm; height ˜150 μm) having 131 recording/stimulating sites as per the Nerve-On-A-Chip® design under one embodiment.

FIG. 14 provides (a) schematic of a shadow mask under one embodiment, (b) a schematic of a micromilled lamination under one embodiment, and (c) a micromilled stainless steel stencil mask under one embodiment.

FIG. 15 shows box plots of N=20 electrodes showing variation in base diameter (left) and height (right).

FIG. 16 provides (a) a photomicrograph of ten micro-porous platinum electrodes of a single patch under one embodiment (b) and a close-up view of the micro-porous platinum electrodes. (c) A photomicrograph of a 3D microtower MEAs prior to electroless plating under one embodiment (d) and a close-up view of the MEAs prior to electroless plating.

### DETAILED DESCRIPTION OF THE INVENTION

### Abbreviations and Definitions

Detailed descriptions of one or more preferred embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification can refer to “one,” but can also refer to “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” can refer to “approximately,” “roughly,” “around,” or “in the region of” When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the terms “microengineered physiological system,” “organotypic preparations,” “3D cellular networks,” “3D organ model” “organs-on-a-chip,” and the like can refer to any biomimetic in vitro system. In embodiments, the microengineered physiological systems are configured to express structural and functional characteristics of a particular biological system. One example of a microengineered system includes a three-dimensional cell culturing system. In one embodiment, the microengineered physiological system comprises a three-dimensional cell culturing system for neural cells that promotes both structural and functional characteristics that mimic those of in vivo nerve fibers. Certain microengineered physiological systems can be configured to promote the growth of isolated cells, tissue explants, tissue explant fragments, or a combination thereof. In embodiments, the microengineered physiological system includes neuronal cells, neural cells, neural tissue explants, or a combination thereof. In embodiments, the microengineered physiological system comprises any of the various systems disclosed in U.S. patent application Ser. No. 15/510,977, the entire contents of which is hereby incorporated by reference. The microengineered physiological system can comprise any of the various systems disclosed in U.S. patent application Ser. No. 16/077,411, the entire contents of which is hereby incorporated by reference.

As used herein, “tissue explants” can comprise any tissue obtained, isolated, or otherwise disassociated from an organism or subject. Exemplary tissue explants include an isolated neural explant. Tissue explants can comprise an explant of any electrically active or electrically responsive tissue. In embodiments, the tissue explant includes an explant of peripheral neural tissue, and explant of central neural tissue, or a combination thereof. An explant can be a brain-derived tissue explant, a spinal cord-derived tissue explant, an enteric-derived tissue explant, a peripheral-derived tissue explant, or a combination thereof. In embodiments, the tissue explant comprises a dorsal root ganglion (DRG) explant, a, a retinal explant, a cortical explant, or a combination thereof. A tissue explant can comprise a plurality of one or more neuronal cells.

The terms “neuronal cells,” “neural cells,” and the like, as used herein can refer to cells that comprise at least one or a combination of dendrites, axons, and somata, or, alternatively, any cell or group of cells isolated from or found within nervous system tissue. In embodiments, neuronal cells are any cell that comprises or is capable of forming an axon. Neuronal cells can comprise isolated primary ganglion tissue. In some embodiments, the neural cell is a Schwann cell, a glial cell, neuroglia, a cortical neuron, an embryonic cell isolated from or derived from neuronal tissue or that has differentiated into a cell with a neuronal phenotype or a phenotype which is substantially similar to a phenotype of a neural cell, induced pluripotent stem cells (iPS) that have differentiated into a neuronal phenotype, or mesenchymal stem cells that are derived from neural tissue or differentiated into a neural phenotype. In certain embodiments, neuronal cells are neurons from dorsal root ganglia (DRG) tissue, retinal tissue, spinal cord tissue, enteric tissue, or brain tissue, in each case from an adult, adolescent, child, or fetal subject. In some embodiments, neural cells are any one or plurality of cells isolated from the neural tissue of a subject. In embodiments, neural cells comprise a primary cell derived from the peripheral nervous system of a subject, a primary cell derived from the central nervous system of a subject, or a combination thereof. In some embodiments, the neural cells are mammalian cells. In embodiments, the cells are human cells. In certain embodiments, the neural cells are derived from primary human tissue or from human stem cells. In some embodiments, the cells are non-human mammalian cells or derived from cells that are isolated from non-human mammals. If isolated or disassociated from the original animal from which the cells are derived, the neuronal cells can comprise isolated neurons from more than one species.

In embodiments, neuronal cells are one or more of the following neurons: sympathetic neurons, spinal motor neurons, central nervous system neurons, motor neurons, sensory neurons, cholinergic neurons, GABAergic neurons, glutamatergic neurons, dopaminergic neurons, serotonergic neurons, interneurons, adrenergic neurons, and trigeminal ganglion neurons. In some embodiments, neural cells are one or more of the following glial cells: astrocytes, oligodendrocytes, Schwann cells, microglia, ependymal cells, radial glia, satellite cells, enteric glial cells, and pituyicytes. In some embodiments, neural cells are one or more of the following immune cells: macrophages, T cells, B cells, leukocytes, lymphocytes, monocytes, mast cells, neutrophils, natural killer cells, and basophils. In some embodiments, neural cells are one or more of the following stem cells: hematopoietic stem cells, neural stem cells, adipose derived stem cells, bone marrow derived stem cells, induced pluripotent stem cells, astrocyte derived induced pluripotent stem cells, fibroblast derived induced pluripotent stem cells, renal epithelial derived induced pluripotent stem cells, keratinocyte derived induced pluripotent stem cells, peripheral blood derived induced pluripotent stem cells, hepatocyte derived induced pluripotent stem cells, mesenchymal derived induced pluripotent stem cells, neural stem cell derived induced pluripotent stem cells, adipose stem cell derived induced pluripotent stem cells, preadipocyte derived induced pluripotent stem cells, chondrocyte derived induced pluripotent stem cells, and skeletal muscle derived induced pluripotent stem cells. In some embodiments, neural cells are keratinocytes. In some embodiments, neural cells are endothelial cells.

The term “isolated neurons,” “isolated neuronal cells,” “isolated neural cells,” and the like can refer to neural cells that have been removed or disassociated from an organism or culture from which they originally grow. In some embodiments isolated neurons are neurons in suspension. In some embodiments, isolated neurons are a component of a larger mixture of cells including a tissue sample or a suspension with non-neuronal or non-neural cells. In some embodiments, neural cells have become isolated when they are removed from the animal from which they are derived, such as in the case of a tissue explant. In some embodiments isolated neurons are those neurons in a DRG excised from an animal. In some embodiments, the isolated neurons comprise at least one or a plurality cells that are from one species or a combination of the species chosen from: sheep cells, goat cells, horse cells, cow cells, human cells, monkey cells, mouse cells, rat cells, rabbit cells, canine cells, feline cells, porcine cells, or other non-human mammals. In some embodiments, the isolated neurons are human cells. In some embodiments, the isolated neurons are stem cells that are pre-conditioned to have a differentiated phenotype similar to or substantially similar to a human neuronal cell. In some embodiments, the isolated neurons are human cells. In some embodiments, the isolated neurons are stem cells that are pre-conditioned to have a differentiated phenotype similar to or substantially similar to a non-human neuronal cell. In some embodiments, the stem cells are selected from: mesenchymal stem cells, induced pluripotent stem cells, embryonic stem cells, hematopoietic stem cells, epidermal stem cells, stem cells isolated from the umbilical cord of a mammal, or endodermal stem cells.

The terms “neuronal cell culture medium” or simply “culture medium” as used herein can refer to any nutritive substance suitable for supporting the growth, culture, cultivating, proliferating, propagating, or otherwise manipulating of cells. In some embodiments, the medium comprises neurobasal medium supplemented with nerve growth factor (NGF). In some embodiments, the medium comprises fetal bovine serum (FBS). In embodiments, the medium comprises L-glutamine. The culture medium can comprise cyclic adenosine monophosphate (cAMP). In certain embodiments, the medium comprises ascorbic acid in a concentration ranging from about 0.001% weight by volume to about 0.01% weight by volume. In embodiments, the medium comprises ascorbic acid in a concentration ranging from about 0.001% weight by volume to about 0.008% weight by volume. In some embodiments, the medium comprises ascorbic acid in a concentration ranging from about 0.001% weight by volume to about 0.006% weight by volume. The medium can comprise ascorbic acid in a concentration ranging from about 0.001% weight by volume to about 0.004% weight by volume. In some embodiments, the medium comprises ascorbic acid in a concentration ranging from about 0.002% weight by volume to about 0.01% weight by volume. In embodiments, the medium comprises ascorbic acid in a concentration ranging from about 0.003% weight by volume to about 0.01% weight by volume. In certain embodiments, the medium comprises ascorbic acid in a concentration ranging from about 0.004% weight by volume to about 0.01% weight by volume. In embodiments, the medium comprises ascorbic acid in a concentration ranging from about 0.006% weight by volume to about 0.01% weight by volume. The medium can comprise ascorbic acid in a concentration ranging from about 0.008% weight by volume to about 0.01% weight by volume. In some embodiments, the medium comprises ascorbic acid in a concentration ranging from about 0.002% weight by volume to about 0.006% weight by volume. In some embodiments, the medium comprises ascorbic acid in a concentration ranging from about 0.003% weight by volume to about 0.005% weight by volume. In embodiments that incorporate Schwann cell differentiation, the culture medium can comprise absorbic acid, FBS, cAMP, or a combination thereof.

The terms “subject” as used herein includes all members of the animal kingdom including, but not limited to, mammals, reptiles, animals (e.g., cats, dogs, horses, swine, primates, rats, mice, rabbits, etc.) and humans.

The term “electrical stimulation” can refer to a process in which the cells are being exposed to an electrical current of either alternating current (AC) or direct current (DC). The current can be introduced into the solid substrate or applied via the cell culture media or other suitable components of the cell culture system. In some embodiments, the electrical stimulation is provided to the device or system by positioning one or a plurality of electrodes at different positions within the device or system to create a voltage potential across the cell culture vessel. The electrodes are in operable connection with one or a plurality of amplifiers, voltmeters, ammeters, and/or electrochemical systems (such as batteries or electrical generators) by one or a plurality of wires. Such devices and wires create a circuit through which an electrical current is produced and by which an electrical potential is produced across the tissue culture system.

The term “solid substrate” as used herein can refer to any substance that is a solid support that is free of or substantially free of cellular toxins. In some embodiments, the solid substrate comprises one or a combination of silica, plastic, and metal. In embodiments, the solid substrate comprises pores of a size and shape sufficient to allow diffusion or non-active transport of proteins, nutrients, and gas through the solid substrate in the presence of a cell culture medium. In certain embodiments, the pore size is no more than about 10, 9, 8, 7, 6, 5, 4, 3, 2, 1 micron microns in diameter. One of ordinary skill could determine the necessary or appropriate pore size based upon the contents of the cell culture medium and exposure of cells growing on the solid substrate in a particular microenvironment. For instance, one of ordinary skill in the art can observe whether any cultured cells in the system or device are viable under conditions with a solid substrate comprises pores of various diameters. In some embodiments, the solid substrate comprises a base with a predetermined shape that defines the shape of the exterior and interior surface. In embodiments, the base comprises one or a combination of silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber; and wherein the opening is positioned at one end of the cylinder. In some embodiments, the base comprises one or a plurality of pores of a size and shape sufficient to allow diffusion of protein, nutrients, and oxygen through the solid substrate in the presence of the cell culture medium. In embodiments, the solid substrate comprises a plastic base with a pore size of no more than 1 micron in diameter and comprises at least one layer of hydrogel matrix; wherein the hydrogel matrix comprises at least a first cell-impenetrable polymer and at least a first cell-penetrable polymer; the base comprises a predetermined shape around which the first cell-impenetrable polymer and at least a first cell-penetrable polymer physically adhere or chemically bond; wherein the solid substrate comprises at least one compartment defined at least in part by the shape of an interior surface of the solid substrate and accessible from a point outside of the solid substrate by an opening, optionally positioned at one end of the solid substrate. In embodiments, where the solid substrate comprises a hollow interior portion defined by at least one interior surface, the cells in suspension or tissue explants can be seeded by placement of cells at or proximate to the opening such that the cells can adhere to at least a portion the interior surface of the solid substrate for prior to growth. The at least one compartment or hollow interior of the solid substrate allows a containment of the cells in a particular three-dimensional shape defined by the shape of the interior surface solid substrate and encourages directional growth of the cells away from the opening. In the case of neuronal cells, the degree of containment and shape of the at least one compartment are conducive to axon growth from soma positioned within the at least one compartment and at or proximate to the opening. In certain embodiments, the solid substrate is tubular or substantially tubular such that the interior compartment is cylindrical or partially cylindrical in shape. In embodiments, the solid substrate comprises one or a plurality of branched tubular interior compartments. In embodiments, the bifurcating or multiply bifurcating shape of the hollow interior portion of the solids is configured for or allows axons to grow in multiple branched patterns. When and if electrodes are placed at to near the distal end of an axon and at or proximate to a neuronal cell soma, electrophysiological metrics, such as intracellular action potential can be measured within the device or system.

In certain embodiments, one or a plurality of electrodes can be placed at or proximate to one or more openings such that recordings can be taken across one or a plurality of positions along an axon length. This can be used to also interrogate one or multiple positions along the length of the axon.

The term “recording” as used herein can refer to measuring the responses of one or more neuronal cells. Such responses can be electro-physiological responses, for example, patch clamp electrophysiological recordings or field potential recordings.

### Microengineered Physiological Systems

For the nervous system, where electrophysiological and histological evaluation are the gold standard measurements to evaluate neuropathies, a biomimetic in vitro system capable of providing clinically-relevant metrics such as nerve conduction velocity and nerve fiber density can improve clinical productivity. A suitable biomimetic in vitro Nerve-On-A-Chip® (NOaC) system has been described in U.S. patent application Ser. No. 15/510,977, the entire contents of which is hereby incorporated by reference. Briefly, embodiments can use animal cells, human cells, or a combination thereof, where axons can be extracellularly stimulated in a 3D polarized structure resulting in unidirectional propagation of signal and thus, evaluation of compound action potentials (CAPs).

### Three-Dimensional (3D) Microelectrode Arrays (MEAs)

While these innovative systems and organotypic preparations provide in vivo information in an in vitro setting, electrophysiological testing included labor-intensive manual placement of stimulating and recording electrodes using micromanipulators which hamper the rate of testing compared to other higher throughput 2D multi-electrode array (MEA) systems.

To overcome this challenge, microengineered physiological systems can be integrated with 3D microelectrodes to automate the process of stimulation, recording, or both. Such automation can increase the throughput of the system making it amenable for screening therapeutic compounds on a large scale. 3D electrodes can interrogate a larger number of diverse axonal fibers to realize population-based electrophysiological responses more akin to in vivo nerve tissue, as compared to other 2D MEA platforms.

Additionally, the planar configuration of conventional MEAs makes them inadequate to capture signals that occur at a certain height when cultures mature to obtain a 3D form. The capture and analysis of signals from thicker, mature tissues is especially important in neurological models on a chip.

Embodiments of the present invention provide a microelectrode design that can be integrated into microphysiological systems such as the 3D hydrogel environment, to permit rapid electrophysiological testing.

Conventional 2D MEA fabrication can involve lithography, metallization, and etching techniques on silicon or glass substrates. Since lithographic techniques on non-planar surfaces is particularly challenging, monolithic 3D MEA fabrication techniques are rare. Recently, there have been tremendous efforts invested into the development of a variety of 3D cell culture systems and as a result, there is a growing need to extend in vitro MEAs to the third dimension. 3D MEAs can permit simple, rapid screening and measurement of network dynamics for the study of 3D microengineered systems for biological systems, including central or peripheral nervous system applications.

3D MEAs can be fabricated on traditional substrates. In embodiments, such traditional substrates comprise any material known in the art to have been commonly used in the construction of microelectrode arrays. Non-limiting examples of traditional substrates include silicon and glass. In alternate embodiments, non-traditional substrates are used in the fabrication of 3D MEAs. Non-traditional substrates include any substrate known in the art to be appropriate for use in fabricating MEAs, but has not historically been used as such. Exemplary non-traditional substrates include, but are not limited to parylene, SU-8, various metals, polyimides, various resins, various epoxies, other non-traditional substrates, or a combination thereof. In one embodiment, silicon-based 3D MEAs are used for in vivo applications. Additionally, metal, glass and polymer probes can be used with 3D MEAs, including 3D MEAs fabricated from technologies such as Electrical Discharge Machining (EDM), polyimide or Kapton micromachining, parylene based technologies, SU-8 based active 3D microscaffold technology with microelectrode and microfluidic functionalities, and Metal Transfer Micromolding (MTM). Fabrication of many of the aforementioned types of 3D MEAs can requires extensive processing in the cleanroom or can involve complex fabrication/assembly methodologies making them expensive and available only to end users with extensive facilities. In addition these technologies can require the investment of significant time to advance from a concept to a final device [Table S1].

For cost-effective and “on demand” manufacturing processes for 3D MEA fabrication, introduction of rapid prototyping technologies utilizing robust, benchtop based, design-to-device strategies is the logical next step. Microfabrication technologies for nanobiosensors, biomedical micro-electro-mechanical systems (BioMEMS) and micro-total analysis systems (MicroTAS) applications have been transitioning away from lithographic techniques towards non-traditional benchtop based fabrication processes as most biological devices do not require the sophistication of the cleanroom environment. A makerspace provides easy access to a variety of tools in an intimidation-free environment to application developers while providing immense flexibility in varied materials and allowing for rapid design changes with scalable fabrication and assembly. We have recently introduced the concept of “Makerspace Microfabrication” which was used for the realization of biological microdevices such as 2D Microelectrode arrays (MEAs), microneedles (MNs) and Microfluidic channels (MFCs). Our ‘Makerspace Microfabrication’ utilizes traditional technologies as needed and has been extended to include new toolbox technologies such as 3D spin cast insulation and electrospinning. In embodiments, the microelectrode arrays disclosed herein can be fabricated using, at least in part, 3D printing, laser etching or micromachining, laserjet or inkjet printing of conductive inks, screen printing, conventional CNC micromilling, electroplating, lamination, or any combination thereof.

In embodiments, ‘Makerspace Microfabrication’ can be used to realize 3D MEAs for electrophysiological assessment of a 3D microengineered system. The process flow for the device can begin with 3D printing to realize the physical structure of the microtowers. In embodiments, 3D microtower MEAs have a base diameter of 250 μm and a height of 400 μm. In various embodiments, the 3D microengineered system can comprise one or more patches, each containing ten recording sites in the form of 3D micro-towers. Certain embodiments comprise two patches. The arrangement of the ten micro-towers can be such that they match with the geometry of the 3D microengineered Nerve-On-A-Chip® which can comprise a circular region (ganglion) leading into a straight channel (neural tract). The micro-towers can overlap both with the circular ganglion and the neural tract to act as recording/stimulating electrodes. A metallization layer, which can be realized by stencil mask evaporation techniques, can define the metallized towers and conductive traces. A biocompatible lamination layer can be used to insulate the traces thereby enabling realization of 3D micro tower MEAs onto which the 3D dual hydrogel constructs for incorporation of dorsal root ganglia (DRG) explants can be defined or transferred. An additional e-beam evaporated SiO2 layer can define a “fine” insulation for the 3D MEA. In applicable embodiments, the metallization and SiO2 evaporation atop 3D printed substrates demonstrates the collaboration between non-traditional and semiconductor processing technologies, which is a characteristic quality of ‘Makerspace Microfabrication’. The hierarchical nature of the process can also allow for subtractive manufacturing techniques such as micromilling and laser micromachining to define the insulation layer. Such a buildup allows for functionalities to be added by every process to realize complex designs. Optical and SEM imaging have been performed to characterize the various constituent processes. Full spectrum impedance analysis of the fabricated electrodes confirms microelectrode nature whose capacitive behavior can be further enhanced by electroless deposition of platinum. Both micro-tower electrodes and smaller 30 μm×30 μm electrodes can be further demonstrated along with chemical and biological characterization of the MEA materials.

In embodiments, the electrodes can comprise any size appropriate for recording or stimulating microengineered physiological systems. In embodiments, at least one of the electrodes comprises a planar electrode of any conceivable shape or form. The shape of the electrode can be elliptical, circular, or polygonal. In embodiments, the shape of the planar electrode comprises a triangle, square, rectangle, rhombus, parallelogram, trapezoid, pentagon, hexagon, heptagon, octagon, nonagon, decagon, circle, oval, half circle, or a quarter circle. The shape of the planar electrode can comprise a curve.

In embodiments, at least one of the electrodes comprises a three-dimensional electrode of any conceivable shape, form, or geometry. In embodiments, the three-dimensional electrode comprises a substantially cylindrical or polyhedral shape. In some embodiments, the three-dimensional electrode comprises a cylindrical pillar, a tapered pillar, or a combination thereof. The three-dimensional electrode can be substantially pyramidal in shape. The three-dimensional electrode can comprise a substantially conical shape.

The present disclosure discloses methods and devices to obtain physiological measurements of microengineered physiological systems including microscale organotypic models of in vitro nerve tissue that mimics clinical nerve conduction and nerve fiber density (NFD) tests. The results obtained from the use of these methods and devices are better predictive of clinical outcomes, enabling a more cost-effective approach for selecting promising lead compounds with higher chances of late-stage success. The disclosure includes the fabrication and utilization of a three-dimensional microelectrode arrays on microengineered system that enables the growth of a uniquely dense, highly parallel neural fiber tract. Due to the confined nature of the tract, this in vitro model is capable of measuring both CAPs and intracellular patch clamp recordings. In addition, subsequent confocal and transmission electron microscopy (TEM) analysis allows for quantitative structural analysis, including NFD. Taken together, the in vitro model system has the novel ability to assess tissue morphometry and population electrophysiology, analogous to clinical histopathology and nerve conduction testing.

Methods of Use

In various exemplary methods, the microelectrode arrays disclosed herein can be employed in microengineered physiological systems to assist with electrophysiological stimulation and recording of electrically active cellular populations.

In various embodiments and through the use of the microelectrode arrays disclosed herein, the present disclosure provides for high-throughput electrophysiological stimulation and recording methods to assess biometric properties of microengineered neural tissue that mimics native anatomical and physiological features. Methods of using the presently disclosed microelectrode arrays provide novel approaches to evaluate neural physiology in vitro, using the compound action potential (CAP) as a clinically analogous metric to obtain results that are more sensitive and predictive of human physiology than those previously available.

One aspect of the present disclosure provides a method for measuring the functions of various cellular targets, including but not limited to, microtubules, ion channels, myelin, mitochondria, and the small nerve fibers. In certain embodiments, the invention includes a method for measuring the myelination of axons using the microelectrode array and the in vitro model described herein. Similar to the structure of a human afferent peripheral nerve, dorsal root ganglion (DRG) neurons in these in vitro constructs project long, parallel, fasciculated axons to the periphery. In native tissue, axons of varying diameter and degree of myelination conduct sensory information back to the central nervous system at different velocities. Schwann cells support the sensory relay by myelinating axons and providing insulation for swifter conduction. Similarly, the three-dimensional growth induced by this in vitro construct comprises axons of various diameters in dense, parallel orientation spanning distances up to 10 mm. Schwann cell presence and sheathing can be observed in confocal and TEM imaging.

Although neuronal morphology is a useful indicator of phenotypic maturity, a more definitive sign of healthy neurons is their ability to conduct an action potential. Apoptosis alone is not a full measure of the neuronal health, as many pathological changes can occur before cell death manifests. Electrophysiological studies of action potential generation can determine whether the observed structures support predicted function, and the ability to measure clinically relevant endpoints produces more predictive results. Similarly, information gathered from imaging can determine quantitative metrics for the degree of myelination, while CAP measurement can demonstrate the overall health of myelin and lends further insight into toxic and neuroprotective mechanisms of various agents or compounds of interest.

As used herein, the “at least one agent” can refer to a small chemical compound. In some embodiments, the at least one agent comprises at least one environmental or industrial pollutant/compound. In certain embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

The at least one agent can comprises one or a combination of chemotherapeutics. Exemplary chemotherapeutics include any one or more of the following: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine (DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide (Oral dacarbazine), Teniposide, Tioguanine (formerly Thioguanine), Topotecan, Tretinoin, Valrubicin, Vemurafenib, Vinblastine Vincristine, Vindesine, Vinorelbine, Vismodegib, and Vorinostat.

In embodiments, the at least one agent comprises one or a combination of analgesics. Exemplary analgesics include, but are not limited to: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators. Cardiovascular modulators can include, but are not limited to: nepicastat, cholesterol, niacin, scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators. Exemplary neuroprotectants and/or neuromodulators include: tryptamine, galanin receptor 2, phenylalanine, phenethylamine, N-methylphenethylamine, adenosine, kyptorphin, substance P, 3-methoxytyramine, catecholamine, dopamine, GABA, calcium, acetylcholine, epinephrine, norepinephrine, and serotonin.

The at least one agent can comprise one or a combination of immunomodulators. Exemplary immunomodulators include: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories. Exemplary anti-inflammatories include: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate.

In certain embodiments, the at least one agent comprises one or a combination of antimicrobials. The antimicrobials can include, but are not limited to: antibacterials, antifungals, antivirals, antiparasitics, heat, radiation, and ozone.

The at least one agent can comprise biological agents or “biologics.” Biologics can refer to any agent or therapeutic that is produced from a living organism or contains a component that is found within living organisms. In embodiments, the “at least one agent” comprises immunoconjugates, small molecule drug conjugates, anti-sense oligonucleotides, nucleic acid therapies, viral vectors, small interfering RNA or a combinations thereof.

In some embodiments, an immunoconjugate can refer to an antibody conjugated to at least one effector molecule or at least one chemical compound. In embodiments, such conjugation can function to increase the efficacy of the antibody molecule for use as a diagnostic or therapeutic agent. Coupling of the antibody with the chemical compound can be accomplished by any mechanism or chemical reaction that binds the two molecules together without affecting the respective activities of the antibody or the chemical compound conjugated thereto. Suitable linking mechanisms include, but are not limited to, covalent binding, affinity binding, intercalation, coordinate binding, complexation, or a combination thereof. In certain embodiments, effector molecules comprise molecules having a desired activity, e.g., cytotoxic activity. Non-limiting examples of effector molecules which can be attached to antibodies include toxins, anti-tumor agents, therapeutic enzymes, radionuclides, antiviral agents, chelating agents, cytokines, growth factors, and oligo- or polynucleotides.

Vectors can include chemical conjugates such as those described in WO 93/64701 (incorporated herein by reference), which has targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618; incorporated herein by reference) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

Vectors can include viral vectors, fusion proteins and chemical conjugates. Retroviral vectors include moloney murine leukemia viruses. Vectors can include pox vectors such as orthopox or avipox vectors, herpesvirus vectors such as a herpes simplex I virus (HSV) vector, adenovirus vectors, and adeno-associated virus vectors.

Pox viral vectors can introduce the gene into the cells cytoplasm. Avipox virus vectors can result in only a short term expression of the nucleic acid. Adenovirus vectors, adeno-associated virus vectors, and herpes simplex virus (HSV) vectors are can introduce the nucleic acid into neural cells. The adenovirus vector can result in a shorter term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaPO4 precipitation, DEAE dextran, electroporation, protoplast fusion, lipofection, cell microinjection, and viral vectors. Vectors can be employed to target essentially any desired target cell.

Another aspect of the present disclosure includes a method of measuring both intracellular and extracellular recordings of biomimetic neural tissue in a three-dimensional culture platform. Previously, electrophysiological experiments were undertaken in either dissociated surface-plated cultures or organotypic slice preparations, with limitations inherent to each method. Investigation in dissociated cell cultures is typically limited to single-cell recordings due to a lack of organized, multi-cellular neuritic architecture, as would be found in organotypic preparations, such as the microengineered physiological systems disclosed herein. Organotypic preparations have intact neural circuitry and allow both intra- and extracellular studies. However, acute brain slices present a complex, simultaneous array of variables without the means to control individual factors and thus are inherently limited in throughput possibility.

The present disclosure provides a biomimetic three-dimensional neural culture that allows examination of population-level electrophysiological behavior. The systems and methods disclosed herein support whole-cell patch clamp techniques and synchronous population-level events in extracellular field recordings resulting from the confined neurite growth in a three-dimensional geometry.

Using the methods and devices disclosed herein, field recordings can be used to measure the combined extracellular change in potential caused by signal conduction in all recruited fibers. The population response elicited by electrical stimulation is a CAP. Electrically evoked population spikes are graded in nature, comprising the combined effect of action potentials in slow and fast fibers. Spikes are single, cohesive events with swift onsets and short durations that are characteristic of CAPs or responses comprised purely of action potentials with quick signal conduction in the absence of synaptic input. The three-dimensional neural constructs disclosed by the present disclosure also support CAPs stimulated from farther distances along the neurite tract or channel, demonstrating the neural culture's ability to swiftly carry signals from distant stimuli much like an afferent peripheral nerve. The three-dimensional neural cultures of the present disclosure support proximal and distal stimulation techniques useful for measuring conduction properties. In various embodiments, the microelectrode arrays disclosed herein are used for stimulation of a microengineered physiological system, recordation of CAPs, or both.

The systems and methods disclosed herein can be used with one or more growth factors that induce recruitment of numerous fiber types, as is typical in nerve fiber tracts. In particular, nerve growth factor (NGF) preferentially recruits small diameter fibers, often associated with pain signaling, as demonstrated in the data presented herein. It has been shown that brain derived neurotrophic factor (BDNF) and neurotrophic factor 3 (NT-3) preferentially support the outgrowth of larger-diameter, proprioceptive fibers. Growth-influencing factors like bioactive molecules and pharmacological agents can be incorporated with electrophysiological investigation to allow for a systematic manipulation of conditions for mechanistic studies. Additional suitable factors include, but are not limited to Forskolin, TGFB-1, GDNF, Glutamax, N2, B27, FBS, Rock inhibitor, ascorbic acid, BSA, and cAMP.

In various exemplary methods, the presently disclosed microelectrode arrays can be used with microengineered physiological systems to study the mechanisms underlying various neurological disorders. By way of example, disclosed herein are methods of studying myelin-compromising diseases and peripheral neuropathies by investigating the effects of known dysmyelination agents, neuropathy-inducing culture conditions, and toxic neuropathy-inducing compounds on the neural cultures. The present disclosure permits conduction velocity to be used as a functional measure of myelin and nerve fiber integrity under toxic and therapeutic conditions, facilitating studies on drug safety and efficacy. The incorporation of genetic mutations and drugs into neural cultures produced using the techniques disclosed herein can enable the reproduction of disease phenomena in a controlled manner, leading to a better understanding of neural degeneration and possible treatment therapies.

The microelectrode arrays can be used to study pathophysiological mechanisms of toxicity, disease, or any agent within any cell population or to study the effects of toxicity, disease, or any agent on any aspect or component of a cell. By way of example, the various embodiments disclosed herein can be applied to study any contents of a cell, cell membranes, or components of cell membranes. Embodiments can be applied to cell organelles, subcellular organelles, cell cytoplasm, structures within the cell membrane, or a combination thereof. Certain embodiments can be applied to study, microtubules, chromosomes, DNA, RNA, mitochondria, ribosomes, Golgi apparatus, lysosomes, endoplasmic reticulum, vacuoles, fragments of any of the foregoing, or other contents or fragmental contents of a cell. Structures within the cell membrane can include any membrane proteins, membrane channels, membrane receptors, or a combination thereof. Embodiments can be applied to studying cellular interactions with the environment.

Another aspect of the present invention includes a medium to high-throughput assay of neurological function for the screening of pharmacological and/or toxicological properties of chemical and biological agents. In embodiments, the agents are cells, such as any type of cell disclosed herein, or antibodies, such as antibodies that are used to treat clinical disease. In embodiments, the agents are any drugs or agents that are used to treat human disease such that toxicities, effects, or neuromodulation can be compared among a new agent which is a proposed mammalian treatment and existing treatments from human disease. In some embodiments, new agents for treatment of human disease are treatments for neurodegenerative disease and are compared to existing treatments for neurodegenerative disease. In the case of multiple sclerosis as a non-limiting example, the effects of a new agent (modified cell, antibody, or small chemical compound) can be compared and contrasted to the same effects of an existing treatment for multiple sclerosis such as Copaxone, Rebif, other interferon therapies, Tysabri, dimethyl fumarate, fingolimod, teriflunomide, mitoxantrone, prednisone, tizanidine, baclofen, or a combination thereof.

In one aspect, the present invention provides methods of replicating, manipulating, modifying, and evaluating mechanisms underlying myelin-compromising diseases and peripheral neuropathies.

In another aspect, the present disclosure includes medium to high-throughput assays of neuromodulation in human neural cells for the screening of pharmacological and/or toxicological activities of chemical and biological agents.

In various aspects, the presently disclosed microelectrode arrays are employed in conjunction with unique microengineered physiological systems, such as 2D and 3D microengineered neural bundles, in combination with optical and electrochemical stimulation to permit recording of human neural cell populations.

Also provided herein are methods of quantifying evoked post-synaptic potentials in a biomimetic, microengineered physiological systems that specifically mimic peripheral neural circuitry, central neural circuitry, or a combination thereof. In embodiments, the microelectrode array is used to study population-level physiology, such as the conduction of compound action potentials and postsynaptic potentials. In certain embodiments any of the various microelectrode arrays disclosed herein can be used to study interactions between separate microengineered physiological systems. By way of example, the microelectrode array can detect interactions between at least two independent organoid systems, between at least two independent organ-on-a-chip systems, between at least one organoid system and at least one organ-on-a-chip system, or a combination thereof.

In another aspect, optogenetic methods, hardware and software control of illumination and fluorescent imaging are used in association with the microelectrode arrays disclosed herein to permit noninvasive stimulation and recording of multi-unit physiological responses to evoked potentials in neural circuits.

Additional methods include the study employing the microelectrode arrays in testing selective 5-HT reuptake inhibitors (SSRIs) and second-generation antipsychotic drugs to see if they alter their developmental maturation.

In another various exemplary embodiments, the microelectrode arrays disclosed herein are used to infer conduction velocity as a functional measure of neural tissue condition under toxic and therapeutic conditions. Information on degree of myelination, myelin health, axonal transport, mRNA transcription, neuronal damage, or a combination thereof can be determined from electrophysiological analysis. Taken in combination with morphometric analysis such as nerve density, myelination percentage, and nerve fiber type, mechanisms of action can be determined for compounds of interest. In some embodiments, the devices, methods, and systems disclosed herein can incorporate genetic mutations and drugs to reproduce disease phenomena in a controlled manner, leading to a better understanding of neurological disorders and possible treatment therapies.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Background: Industrial pollutants and other toxins that can make their way into our environment are well known to cause neurotoxicity in humans. Peripheral neuropathy is one of the most common responses of the nervous system to chemical toxicity [1]. This may be because peripheral nerve axons extend long distances from their cell bodies lying outside the protective blood-brain barrier. Various toxins can produce cytotoxicity of the cell bodies, demyelination, or distal axonal degeneration. In humans, symptoms of sensory loss in the hands and feet usually occur before noticeable motor weakness [1]. Experimental models that recapitulate these pathological phenomena would be the most useful for screening chemicals for toxicity, identifying toxic mechanisms, and evaluating therapeutic countermeasures.

Since the inception of the Tox21 program in 2008, a variety of in vitro quantitative high throughput screening (qHTS) assays have been developed to screen thousands of compounds in a relatively short period of time [2, 3]. While animal testing provides more specific anatomical and physiological parameters after chemical exposure, the cost and time of performing such experiments precludes their use for screening thousands of compounds [4, 5]. On the other hand, while qHTS assays are rapid and inexpensive in nature, they typically only provide one or two biological outputs at a time [3] and lack in providing parameters which closely relate to in vivo metrics. This mismatch is especially true for the nervous system, where clinically-relevant metrics such as nerve conduction velocity and histological analysis are considered as gold standard [6] [7]. Quantitative HTS assays [8] and development of 2D multielectrode arrays (MEA) [9] have bridged the gap slightly but they are still incapable of providing metrics which are comparable to in vivo systems.

Microphysiological systems (MPS) or “organoid-on-a-chip” models (OCM) show tremendous promise as advanced cellular models that can provide medium-throughput and high-content data useful for toxin screening, provided that they supply information that is predictive of organism physiology or pathology. The NIH tissue chips program (ncats.nih.gov/tissuechip) has enhanced the pace of development of MPS for drug safety and efficacy testing. However, the main focus of this program is development of only human tissue chips where validation is challenging because of the lack of clinical data for the majority of untested chemical toxins. Thus, development of 3D organotypic cellular models utilizing animal cells is important for the validation of these systems by direct comparison to animal data, which should be undertaken before assuming that human tissue chips predict clinical drug safety and efficacy.

A number of contract research organizations have seen commercial success providing such assays for various organ systems. However, development of peripheral Nerve-On-A-Chip® assays is lagging. Commonly-used peripheral neural culture preparations are not predictive of clinical toxicity, partially because they typically utilize apoptosis or neurite elongation as measurable endpoints, whereas adult peripheral neurons are fully grown and known to resist apoptosis. Nerve conduction testing and histomorphometry of tissue biopsies are the most clinically-relevant measures of neuropathy. Nevertheless, there are currently no culture models that provide such metrics. Various brain MPS system have been prepared over years such as 3D neural constructs [10], cerebral organoids [11] or neurospheres [12] to assess neurotoxicity but none of them recapitulated the biomimetic complexity of the nervous system especially peripheral nervous system. MPS that seek to recapitulate the most relevant anatomic and physiological toxic pathology in a simple model require a stronger focus on system architecture [13, 14].

3D Assays Capable of Detecting Compound Action Potentials Hold the Promise of Bridging the Gap Between Ex Vivo to In Vivo Animal Toxicity Screening.

We have developed a sensory-Nerve-On-A-Chip® model by culturing dorsal root ganglia in micropatterned hydrogel constructs to constrain axon growth in a 3D arrangement analogous to peripheral nerve anatomy. Further, electrically-evoked population field potentials resulting from compound action potentials (CAPs) can be recorded reproducibly in these model systems. These early results demonstrate the feasibility of using microengineered neural tissues that are amenable to morphological and physiological measurements analogous to those of animal (and clinical) tests. From a single in vitro preparation, we can measure CAP amplitude and conduction velocity, and then subsequently section the tissue to measure histomorphological parameters such as axon diameter, axon density, and myelination. We hypothesize that this 3D organotypic system is capable of detecting neural toxicity parameters in ways that mimic clinical neuropathology. This versatile system could also further be used for performing “omics” studies and thus will eventually be used for determining a large spectrum of toxicological parameters resulting in understanding mechanisms of action as well as improved understanding of biological processes.

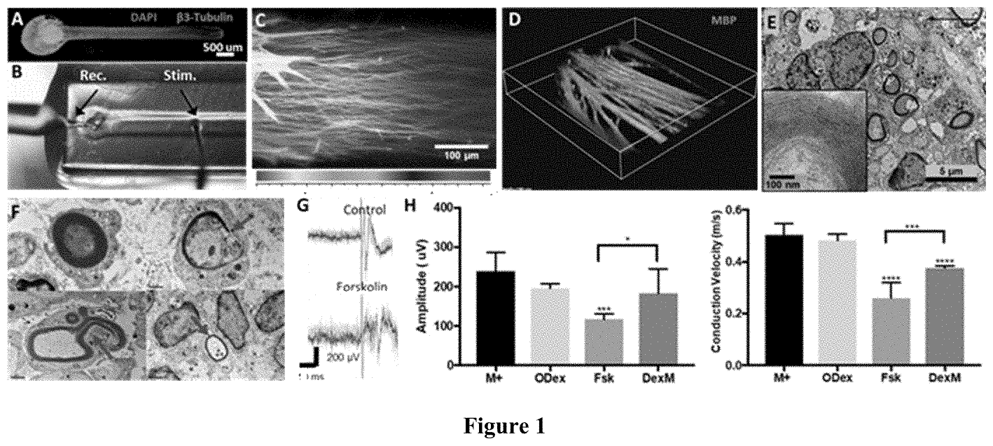

We have developed a simple but unique method of digital projection lithography for rapid micropatterning of one or more hydrogels directly onto conventional cell culture materials [15]. Our simple and rapid approach uses two gels: polyethylene glycol (PEG) as a restrictive mold, and crosslinked methacrylated gelatin (Me-Gel) as a permissive matrix. These dual gels constrain neurite growth from embryonic dorsal root ganglion (DRG) explants within a particular 3D geometry, resulting in axon growth with high density and fasciculation (FIGS. 1C &D). When cultured in myelin induction medium, we observe a tremendous degree of myelin staining positive for myelin basic protein (MBP), indicating compact myelin (FIG. 1D), whose characteristic spiral structure is evident from TEM images (FIG. 1E). This unique culture model, with a highly-parallel biomimetic 3D neural fiber tract, corresponds to peripheral nerve architecture; it may be assessed using neural morphometry, allowing for clinically-analogous assessment unavailable to traditional cellular assays. Most unique to our Nerve-On-A-Chip® model is the ability to record compound action potentials (CAPs). Traces show characteristic uniform, short-latency population responses that remain consistent with high frequency (100 Hz) stimulation and can be reversibly abolished by tetrodotoxin, and the responses are insensitive to neurotransmitter blockers, indicating CAPs rather than synaptic potentials [16].

As an example of how neurological effects of compounds may be evaluated in this model system, we administered a 48-hr application of 40 μM forskolin (Fsk), which has been shown to cause dysmyelination in vitro [17]. In our model system, histology indicated strong evidence of dysmyelination (FIG. 1F), resulting in 70% reduction in % myelinated axons and 50% increase in G-ratio, indicating thinning of myelin sheath (data not shown). Electrophysiology indicated about a 50% reduction in both CAP amplitude and conduction velocity (FIG. 1G). Co-administering dexamethasone (DexM) partially restored these effects (FIG. 1H). These morphological and physiological measurements are analogous to the most powerful metrics available in animal models of peripheral nerve pathology [18].

Embryonic DRG cultures have been used effectively as models of peripheral nerve biology for decades [19]. While useful as model systems, conventional DRG cultures are known to be poorly predictive of clinical toxicity when assessed with traditional cell death assays. While single-cell recordings may be obtained from DRGs, we are aware of no reports of recording CAPs, due to the lack of tissue architecture. Unlike prior model systems, the presently disclosed system includes the ability to assess tissue morphometry and population electrophysiology, analogous to clinical histopathology and nerve conduction testing.

Without being bound by theory, chemical toxins known for causing neurotoxicity in rats will induce toxicity in microengineered neural tissue as quantified using morphological and physiological measures analogous to clinical metrics. We will approach this objective by first enhancing the throughput of our system by engineering 3D microelectrodes to test electrophysiological characteristics of the model system. Next, we will determine baseline variability and characterize structure-function relationships using the 3D microelectrodes. We will then quantify changes induced by acute application of chemical toxins to demonstrate the technical merit of using the compound action potential (CAP) waveform as a preclinical assay of neurotoxicity.

Develop a Custom 3D Microelectrode Array for Quantification of Progressive Neurodegeneration in 3D Microengineered Rat Peripheral Nerve Tissue.

Subtask 1.1—Develop and Test 3D Microelectrodes Configured for Peripheral Nerve-On-A-Chip®.

Three-dimensional microelectrode arrays (3D MEAs) represent the next generation of tools for interrogating a variety of cell cultures, biomaterials and other biological agents ex vivo. These tools can impart the necessary complexity required to reduce animal testing and improve the efficacy of cell-based biosensors for a variety of applications including our targeted Nerve-On-A-Chip®. Typically, 3D MEAs are fabricated in plane and assembled out of plane [20, 21] or defined monolithically [22-24] out of glass or silicon wafers. The assembly is typically performed using chip-on-board technologies. However, the cost-effective fabrication and system-level assembly of these arrays presents significant challenges and possibilities for new innovations. For 3D applications, metal “bulbs” [25] and carbon nanotubes [26] or nanopillars [27] have been demonstrated for potential recording in the 3D volume of tissue, though repeatable demonstration of high SNR recordings remains a challenge. We will develop at least two innovations: (1) All-polymer, package and device co-fabrication of 3D MEAs with controllable 3D location of the microelectrode fabricated utilizing “makerspace” microtechnologies; (2) 3D nanotextured volumetric materials for 3D electrodes defined arbitrarily on the MEA that can be employed in volumetric stimulation.

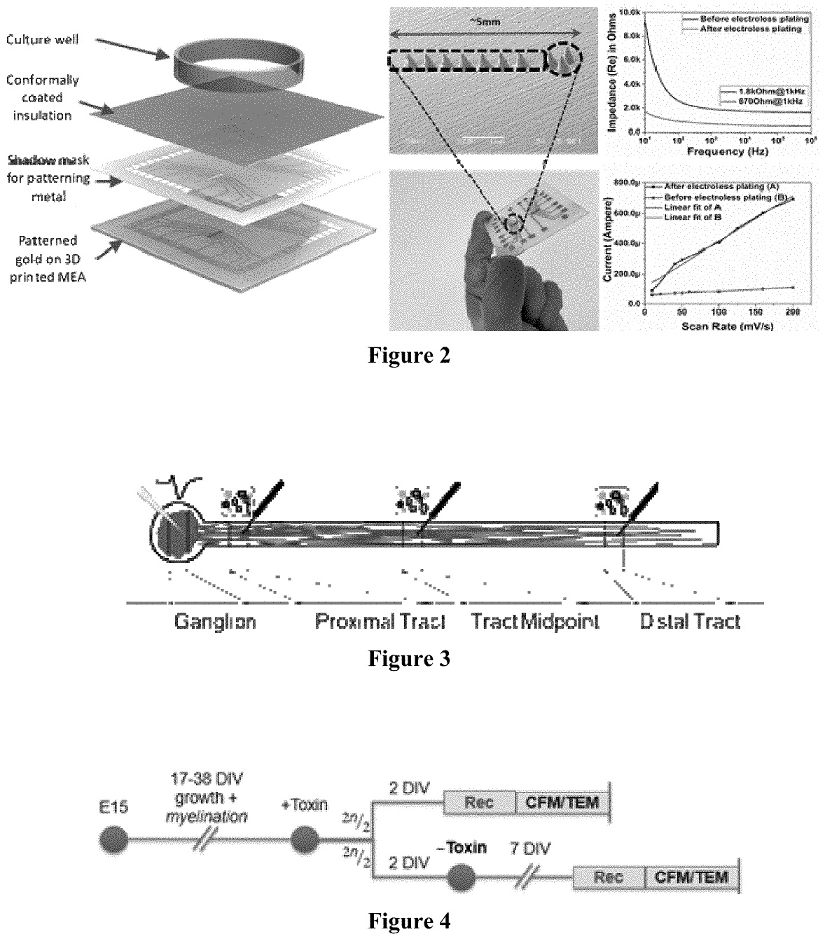

Methods: FIG. 2 depicts a schematic illustration of the microfabrication process flow that developed for 3D MEAs. The chip can accommodate 16 microneedle-type electrodes (3D with various heights from 300-1000 μm) and 16 planar electrodes in the 5 mm×500/800 μm area of the nerve-on-a chip platform. The overall size of the chip will be 49 mm²×1 mm to interface with the commercial MultiChannel Systems recording amplifiers. We will utilize 3D printing to develop the structural features of such a device in a single step. We have made tremendous progress in utilizing 3D printing technology recently for the development of biomedical devices [28]. The technology represents advantages such as rapid translation from design to a final device, ability to define complex structures such as microfluidic ports, and development of a variety of arrays with different dimensions all in a single process step. Co-design and co-fabrication of device and package and lastly choice of various biocompatible resin materials can be utilized in the printing process. Shadow/stencil masks will be fabricated from suitable metals or polymers (e.g. stainless steel or Kapton) utilizing CNC micromilling (T-Tech QC J-5) and/or laser micromachining. CNC micromilling can be used to define patterns down to ˜7 μm with nano-milling tools. Alternatively, these stencil masks can be fabricated with a laser micromachining tool (EzLaze 3) which is multimodal (with wavelengths 1064 nm, 355 nm and 532 nm respectively) and can be used to define the patterns for the shadow mask on a variety of materials such as polymers, metals and resins down to ˜1 μm. Subsequent metallization with a layer of Titanium/Gold will be performed to define the metal on the electrodes, high density metal tracks and package bond pads. Matching the thermal and mechanical properties of the shadow mask and the 3D printing resin is vitally important for shadow mask metallization as is the critical need for alignment features. Multi-layer processing, for instance top and bottom side metallization on the 3D printed resin followed by screen printing (ASYS XM Manual Printer) of conductive inks in vias defined by 3D printing for interconnectivity can further be utilized for increasing the density of the 3D and 2D electrodes.

The ink will be cured in an oven to achieve its final properties. Several biocompatible inks are available for such a purpose and these will be tailored to the intended application for resistivity, surface porosity and ease of fabrication. We will ascertain these properties during process development with tools such as a SEM and an AFM. Such feedback to process development is critical to achieve the desired characteristics of the metal traces and the conductive vias. The final insulation on the defined conductive ink and metal electrodes needs to be deposited and recording sites defined in planar and in the third dimension for the creation of the electrodes. Parylene is an ideal insulation layer because it is biocompatible, can be conformally coated at room temperature, and is laser micromachinable [29]. Parylene will be deposited (SCS Lab Coater) on the arrays and the recording sites at arbitrary 3D locations will be defined utilizing laser micromachining in the UV mode.

We have already fabricated test devices in configurations that are compatible with both the Nerve-On-A-Chip® tissue architecture as well as with off-the-shelf MEA recording equipment (FIG. 2). Samples have been provided for testing and feedback. Microelectrode noise is a characteristic that determines the ability of the electrode to pick up or deliver small current or voltage signals from 2D and 3D neural cultures. Additionally, 3D nanomaterials defined as volumetric stimulators can perform stimulation of tissue in the Nerve-On-A-Chip® in all three dimensions creating interesting responses. We have extensive experience in the development of low impedance and biocompatible nanomaterials such as nano-porous platinum or electroplated/nano-textured gold on the electrode sites [29, 30]. Optimized gold electroplating and nanotexturing (EzLaze 3) recipes will be developed for both 2D surface conditioning of the electrodes and defining 3D volumetric stimulators with over-plating techniques. These methods will be evaluated directly using SEM, AFM and indirectly with the measurement of noise using the MultiChannel Systems amplifier. Such techniques can be adapted to realize low noise electrodes that are 30-50 μm in diameter or smaller.

The 3D MEAs will be evaluated for electronic, electrochemical, and electrophysiological performance. For electronic characterization of 3D microelectrodes and comparison to 2D microelectrodes, full-spectrum impedance performance of the devices establishes key application related electrode characteristics and additionally provides feedback to micro/nanofabrication. We will use physiological saline and a reference platinum wire electrode to test impedance characteristics of the MEA. A full spectrum impedance measurement from 1 Hz to 10 MHz will be performed utilizing the BODE impedance analyzer and the 2D and 3D electrodes will be compared utilizing this technique and process yields of the fabrication processes estimated. Additionally, the impedance data from our electrode nanomaterials can be compared to literature and electrode geometries will be tailored for the Nerve-On-A-Chip® application. For electrochemical characterization of the two types of electrodes, cyclic voltammograms (CV) that quantify the charge-carrying capacity of individual microelectrode materials will be measured using eDAQ potentiostat and compared to the voltammograms from commercial thin film gold electrodes from literature. These metrics enable reliability of the electrodes for long term use, ensure stimulation capabilities and lifetime measurements for the electrode nanomaterials.

Subtask 1.2: Characterize the Biological Performance of 3D MEAs Using Dual Hydrogel Constructs and Embryonic Rat DRG Tissue.

Without being bound by theory, the 3D electrode design will address the unique tissue architecture of the Nerve-On-A-Chip® and enable sampling relatively large tissue volumes for NCV testing, which in humans has been shown to predict the type and severity of clinical nerve pathology even before symptoms fully manifest [31]. Growing tissue on top of 3D MEA will increase the throughput of our Nerve-On-A-Chip® by automating the electrophysiological testing and enable us to chronically measure the neurodegeneration over weeks from the same construct. This is currently not practical with conventional field electrodes.

Methods: For biological characterization, 3D MEA devices will be incorporated with hydrogel-tissue constructs and electrophysiological evaluation. The projection lithography method we have pioneered makes it possible to pattern hydrogel substrates on numerous surfaces [15, 16]. Myelinated and unmyelinated neural tissue constructs will be fabricated using improvements on our published work [15, 16]. Dual hydrogel constructs will be fabricated from PEG gel micromolds filled with methacrylated gelatin (Me-Gel) supplemented with laminin. Neurite growth constructs will be fabricated to be ˜400 μm wide and up to 10 mm in length (FIGS. 1A&B). Dorsal root ganglia (DRG) will be taken from thoracic levels of spinal cords dissected from embryonic day 15 (E15) rat embryos and incorporated within bulbar regions of the dual hydrogel constructs. Myelinated tissue constructs will be cultured for 10 days in Basal Eagle's Medium with ITS supplement and 0.2% BSA to promote Schwann cell migration and neurite outgrowth, followed by culture for up to four more weeks in the same medium additionally supplemented with 15% FBS and 50 μg/ml ascorbic acid to induce myelination [32]. Unmyelinated constructs will be formed by culturing in the same media regimen, but lacking ascorbic acid. At least two weeks of culture in myelin induction medium, with ascorbic acid, is required for substantial formation of compact myelin.

MEAs will be inserted into commercial recording equipment and tissue will be stimulated at 7 different locations along axon growth region, while recording will be taken simultaneously from 3 recording locations corresponding to ganglion region (FIG. 3). Compound action potentials (CAPs) will be considered measurable if amplitudes reach 50 μV or more. To assess tissue morphology at various stages of maturity, approximately 12 each of myelinated and unmyelinated tissue constructs will be fixed in 4% paraformaldehyde at one, two, three, and four weeks in myelination induction medium (or 17, 24, 31, and 38 total days in vitro, DIV) and stained for nuclei (Hoechst), neurites Schwann cells (S-100), myelin basic protein (MBP), and apoptosis (Annexin-V and TUNEL). Samples will be imaged with confocal microscopy at regions within DRG, proximal to the ganglion, near midpoint of fiber tract, and in the fiber tract distal to the ganglion; exact distances will be proportional to average maximal neurite extent.

TABLE 1
Morphological and physiological metrics.
Morphology

| | |
|---|---|
| Cell body density | Axon density |
| Diameter distr. | Axon diameter distribution |
| | % Myelinated axons |
| | Myelin thickness distribution |

Physiology

| | |
|---|---|
| % Apoptotic cells | Amplitude distribution |
| | Envelope distribution |
| | Area under curve distribution |
| | Conduction velocity distribution |

Potential Alternatives. Development of 3D MEAs in a Nerve-On-A-Chip® platform is not a trivial problem. Despite tremendous advances in 3D printing technologies made recently, the resolution of our 3D Printer is approximately 100 μm with a single layer cure of 25 μm. In order to increase the density of the 3D and 2D microelectrodes or if problems arise in the primary fabrication process in the 5 mm×500/800 μm area of the Nerve-On-A-Chip® device, we will utilize the well-characterized combination of Metal Transfer Micromolding and laser micromachining technologies [29, 30] to fabricate the MEA chip with 32 electrodes and increasing the density to 64 electrodes. These devices will be developed on polymers such as Cyclic Olefin Co-Polymer (COC), Poly Methyl Methacrylate (PMMA) or Polycarbonate (PC). MTM technology has previously been well characterized and studied by the subcontract PI [30, 33, 34]. Separately PCBs will be designed and fabricated from commercial vendors such as Innovative Circuits. The PCB and the MEA chip will be combined utilizing a self-alignment scheme involving an acrylic spacer and a force contact. Parylene can further be deposited on the entire MEA assembly and recording sites defined utilizing laser micromachining.

Interpretation of anticipated results. Without being bound by theory, 3D MEAs will increase the throughput of our Nerve-On-A-Chip® system. Long-term, repeated monitoring of CAP waveforms and NCV will demonstrate baseline physiological parameters for these constructs. Without wishing to be bound by theory, distal amplitudes will appear and increase as neurites elongate past electrodes, and conduction velocity to increase as myelin forms.

Demonstrate the Feasibility of Quantifying Peripheral Neurotoxicity by NCV and Histomorphometry in a 3D Peripheral Nerve-On-A-Chip®.

Studying alterations in complex physiology and unique morphology of the nervous system is a significant challenge while screening neurotoxicants [35]. The number of neurotoxic compounds causing developmental and adult toxicity are rising [36, 37] and traditional in vivo screening as well as in vitro models are still inadequate to screen chemical exposure. AxoSim's 3D organotypic rat model is capable of bridging the predictivity, complexity and throughput of in vivo and in vitro models, while enabling historical benchmarking. To enable a manageable scope, we will restrict experiments to four known chemical toxins acrylamide [38], methylmercury [39], n-hexane (in the form of the metabolite 2,5 hexane dione) [40] and rotenone [41] which have historically demonstrated NCV changes in rats (Table 2). Quasi-3D nature of the micropatterned cultures is amenable to conventional cellular and molecular assays.

TABLE 2
Drug doses for initial pilot study.

| | In Vitro | | Reference |
|---|---|---|---|
| | Neurotoxic | Reference | (Rat study- |
| Chemical | dose | (in vitro) | NCV Testing) |
| Acrylamide | 0.8 mM | [42] | [38] |
| Methylmercury | 10 mM | [43] | [39] |
| n-hexane | 765 mM | [44] | [40] |
| Rotenone | 30 nM | [45] | [41] |

Subtask 2.1: Determine Dosages and Incubation Times in a Pilot Study Involving a Small Library of Compounds with Relevance to Environmental and Industrial Neurotoxicity.

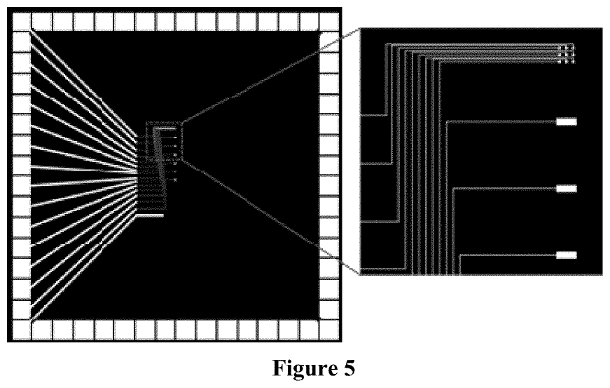

We will first perform a pilot study to ensure effective dosing. We will start with acute (48-hr) doses proven to induce neuronal cell death in vitro (48-hr) and verify that morphological and physiological changes are measurable in our model at these concentrations (FIG. 4).

Methods: DRG explants (n=20) will be cultured in micropatterned gels (as in subtask 1.2) according to myelination induction regimen. At a timepoint determined in subtask 1.2 to produce fully myelinated constructs, specimens will be checked for neurite growth (Cell Tracker Green) and myelination (FluoroMyelin Red); specimens without sufficient neurite growth and/or myelination at this point will be excluded.

Electrophysiological recordings of healthy constructs will be taken, and the next day, neurotoxic concentrations of the four chemicals will be applied for 48 hours, as summarized in Table 2. Controls will receive vehicle only. Electrophysiology will be performed on half (n=10) of explants at the end of the 48-hr administration period, and the other half 7 days after administration period. All specimens will be fixed after final recording, stained, and assessed as summarized in Table 1. NCV data obtained in the 3D system will be compared to NCV data available in the literature as referenced in Table 2. Additionally, qualitative observations will be made of soma and axon damage, such as chromatin condensation, blebbing, and axon segmentation.

Subtask 2.2: Measure CAP Conduction Velocity, Amplitude, Integral, and Excitability after Compound Administration at End Points Determined in Pilot Study and Correlate to Morphometric Changes.

Without being bound by theory, acute administration of each chemical will induce toxicities detectable by measuring changes in CAPs with respect to baseline. Depending on the mechanism, we expect to see changes in CAP amplitude and/or NCV. Subsequent histopathological analysis will provide important quantitative metrics of morphological variability for correlation with physiology. Histopathological analysis is more labor intensive but provides mechanistic details of neurodegeneration [7]. Therefore, understanding the correlation between both metrics can reduce the time and effort required to understand the manifestation and progression of neuropathy. Without being bound by theory, the physiological changes will parallel documented in vivo and clinical pathology.

Methods: Electrophysiological and histological methodology will be identical to subtasks 1.2 and 2.1. After confocal imaging, samples will be post-fixed in 2% osmium tetroxide, dehydrated, and embedded in epoxy resin. ˜10 ultrathin cross-sections will be cut at each defined region (i.e. ganglion, proximal, midpoint, distal) and stained with lead citrate and uranyl acetate for TEM imaging. Analysis will be assessed as summarized in FIG. 3 and Table 1. We will perform statistical cross-correlation to determine which morphological measures best correlate with which physiological measures [46]. Additionally, these experiments will provide measures of variability used for a statistical power analysis to determine appropriate sample sizes for Aim 2, and will be used to define exclusion criteria, e.g. samples with neurite growth more/less than 2 standard deviations from average will be excluded.

Potential alternatives. While the neurotoxicity of the four toxins has been observed in vitro, the biological effects may be influenced by the 3D preparation in unpredictable ways. It is possible that the morphological and physiological pathology expected will not manifest in the pilot study or cell death will overwhelm functional measures. If so, we may increase/decrease the dose and/or switch to a chronic application (7 days). Neuropathy could be evident but quantitative variability could make 10% detectable differences impractical. If so, we will design the larger study to detect a 20%-30% detectable difference.

Interpretation of anticipated results. Without being bound by theory, acute administration of each chemical will induce toxicities that may be detected by measuring changes in CAPs with respect to baseline. We expect most of these changes will correlate with any morphological damage as quantified by our morphometric analysis.

Milestones. 1) Development of 3D microelectrodes capable of real-time, reliable detection CAP of 50 μV or more for several weeks, before and after chemical exposure. 2) Demonstration of the feasibility of AxoSim's Nerve-On-A-Chip® platform to assess the electrophysiological and histological neurotoxicity caused by 4 chemical toxins. These studies represent the basis for further validation studies for comparison to historical in vivo data.

### REFERENCES CITED IN THIS EXAMPLE

1. Ludolph, A. C. and P. S. Spencer, Toxic neuropathies and their treatment. Baillieres Clin Neurol, 1995. 4(3): p. 505-27.
2. Merrick, B. A., R. S. Paules, and R. R. Tice, Intersection of toxicogenomics and high throughput screening in the Tox21 program: an NIEHS perspective. International Journal of Biotechnology, 2015. 14(1): p. 7-27.
3. Tice, R. R., et al., Improving the Human Hazard Characterization of Chemicals: A Tox21 Update. Environmental Health Perspectives, 2013. 121(7): p. 756-765.
4. Sun, H., et al., Paradigm Shift in Toxicity Testing and Modeling. The AAPS Journal, 2012. 14(3): p. 473-480.
5. Shukla, S. J., et al., The future of toxicity testing: a focus on in vitro methods using a quantitative high-throughput screening platform. Drug Discovery Today, 2010. 15(23): p. 997-1007.
6. Virginia, C. M., Functional Assays for Neurotoxicity Testing. Toxicologic Pathology, 2010. 39(1): p. 36-45.
7. Joseph, C. A., S. L. Mona, and G. Z. Elena, Correlation and Dissociation of Electrophysiology and Histopathology in the Assessment of Toxic Neuropathy. Toxicologic Pathology, 2010. 39(1): p. 46-51.
8. Pei, Y., et al., Comparative neurotoxicity screening in human iPSC-derived neural stem cells, neurons and astrocytes. Brain Research, 2016. 1638(Part A): p. 57-73.
9. van Vliet, E., et al., Electrophysiological recording of re-aggregating brain cell cultures on multi-electrode arrays to detect acute neurotoxic effects. Neurotoxicology, 2007. 28(6): p. 1136-1146.
10. Schwartz, M. P., et al., Human pluripotent stem cell-derived neural constructs for predicting neural toxicity. Proceedings of the National Academy of Sciences, 2015. 112(40): p. 12516-12521.
11. Dakic, V., et al., Short term changes in the proteome of human cerebral organoids induced by 5-MeO-DMT. Scientific Reports, 2017. 7(1): p. 12863.
12. Moors, M., et al., Human Neurospheres as Three-Dimensional Cellular Systems for Developmental Neurotoxicity Testing. Environmental Health Perspectives, 2009. 117(7): p. 1131-1138.
13. Astashkina, A. and D. W. Grainger, Critical analysis of 3-D organoid in vitro cell culture models for high-throughput drug candidate toxicity assessments. Advanced Drug Delivery Reviews, 2014. 69-70: p. 1-18.
14. Lelièyre, S. A., T. Kwok, and S. Chittiboyina, Architecture in 3D cell culture: An essential feature for in vitro toxicology. Toxicology in Vitro, 2017. 45: p. 287-295.
15. Curley, J. L. and M. J. Moore, Facile micropatterning of dual hydrogel systems for 3D models of neurite outgrowth. J Biomed Mater Res A, 2011. 99(4): p. 532-43.
16. Huval, R. M., et al., Microengineered peripheral Nerve-On-A-Chip® for preclinical physiological testing. Lab Chip, 2015. 15(10): p. 2221-32.
17. Zhu, T. S. and M. Glaser, Neuroprotection and enhancement of remyelination by estradiol and dexamethasone in cocultures of rat DRG neurons and Schwann cells. Brain Research, 2008. 1206: p. 20-32.
18. Bradbury, A. M., et al., Clinical, electrophysiological, and biochemical markers of peripheral and central nervous system disease in canine globoid cell leukodystrophy (Krabbe's disease). J Neurosci Res, 2016. 94(11): p. 1007-17.
19. Melli, G. and A. Hoke, Dorsal Root Ganglia Sensory Neuronal Cultures: a tool for drug discovery for peripheral neuropathies. Expert Opin Drug Discov, 2009. 4(10): p. 1035-1045.
20. Bai, Q., K. D. Wise, and D. J. Anderson, A high yield microassembly structure for three-dimensional microelectrode arrays. Ieee Transactions on Biomedical Engineering, 2000. 47(3): p. 281-289.
21. Frey, O., et al., Biosensor microprobes with integrated microfluidic channels for bi-directional neurochemical interaction. Journal of Neural Engineering, 2011. 8(6).
22. Campbell, P. K., et al., A Silicon-Based, 3-Dimensional Neural Interface-Manufacturing Processes for an Intracortical Electrode Array. Ieee Transactions on Biomedical Engineering, 1991. 38(8): p. 758-768.
23. Heuschkel, M. O., et al., A three-dimensional multi-electrode array for multi-site stimulation and recording in acute brain slices. Journal of Neuroscience Methods, 2002. 114(2): p. 135-148.
24. Takei, K., et al., Integration of out-of-plane silicon dioxide microtubes, silicon microprobes and on-chip NMOSFETs by selective vapor-liquid-solid growth. Journal of Micromechanics and Microengineering, 2008. 18(3).
25. Hai, A. and M. E. Spira, On-chip electroporation, membrane repair dynamics and transient in-cell recordings by arrays of gold mushroom-shaped microelectrodes. Lab Chip, 2012. 12(16): p. 2865-73.
26. Greenbaum, A., et al., One-to-one neuron-electrode interfacing. J Neurosci Methods, 2009. 182(2): p. 219-24.
27. Sileo, L., et al., Electrical coupling of mammalian neurons to microelectrodes with 3D nanoprotrusions. Microelectronic Engineering, 2013. 111: p. 384-390.
28. Ausaf, T., A. Kundu, and S. Rajaraman, 3-D Printing, Ink Casting, and Lamination (3-D PICL): A rapid, robust, and cost effective process technology toward the fabrication of microfluidic and biological devices, in MicroTAS 20172017, The 21st International Conference on Miniaturized Systems for Chemistry and Life Sciences: Savanah, Ga.
29. Rajaraman, S., et al., Microfabrication technologies for a coupled three-dimensional microelectrode, microfluidic array. Journal of Micromechanics and Microengineering, 2007. 17(1): p. 163-171.
30. Rajaraman, S., et al., Metal-Transfer-Micromolded Three-Dimensional Microelectrode Arrays for in-vitro Brain-Slice Recordings. Journal of Microelectromechanical Systems, 2011. 20(2): p. 396-409.
31. Velasco, R., et al., Early predictors of oxaliplatin-induced cumulative neuropathy in colorectal cancer patients. J Neurol Neurosurg Psychiatry, 2014. 85(4): p. 392-8.
32. Eshed, Y., et al., Gliomedin mediates Schwann cell-axon interaction and the molecular assembly of the nodes of Ranvier. Neuron, 2005. 47(2): p. 215-29.
33. Allen, M., et al., Method for Making Electrically Conductive Three-Dimensional Structures, USPTO, Editor 2007: USA.
34. Rajaraman, S., et al., Micromachined three-dimensional electrode arrays for transcutaneous nerve tracking. Journal of Micromechanics and Microengineering, 2011. 21(8).
35. Bal-Price, A. and H. T. Hogberg, In Vitro Developmental Neurotoxicity Testing: Relevant Models and Endpoints, in In Vitro Toxicology Systems, A. Bal-Price and P. Jennings, Editors. 2014, Springer New York: New York, N.Y. p. 125-146.
36. Grandjean, P. and P. J. Landrigan, Developmental neurotoxicity of industrial chemicals. The Lancet, 2006. 368(9553): p. 2167-2178.
37. Grandjean, P. and P. J. Landrigan, Neurobehavioural effects of developmental toxicity. The Lancet Neurology. 13(3): p. 330-338.
38. Zhu, Y. J., et al., Effects of acrylamide on the nervous tissue antioxidant system and sciatic nerve electrophysiology in the rat. Neurochem Res, 2008. 33(11): p. 2310-7.
39. Chuu, J.-J., S.-H. Liu, and S.-Y. Lin-Shiau, Differential neurotoxic effects of methylmercury and mercuric sulfide in rats. Toxicology Letters, 2007. 169(2): p. 109-120.
40. Takeuchi, Y., et al., A comparative study on the neurotoxicity of n-pentane, n-hexane, and n-heptane in the rat. British Journal of Industrial Medicine, 1980. 37(3): p. 241.
41. Binienda, Z. K., et al., Chronic exposure to rotenone, a dopaminergic toxin, results in peripheral neuropathy associated with dopaminergic damage. Neuroscience Letters, 2013. 541(Supplement C): p. 233-237.
42. Hideji, T. and H. Kazuo, In vitro neurotoxicity study with dorsal root ganglia for acrylamide and its derivatives. Toxicology Letters, 1991. 58(2): p. 209-213.
43. Ali, S. F., C. P. LeBel, and S. C. Bondy, Reactive oxygen species formation as a biomarker of methylmercury and trimethyltin neurotoxicity. Neurotoxicology, 1992. 13(3): p. 637-648.
44. Gartlon, J., et al., Evaluation of a proposed in vitro test strategy using neuronal and non-neuronal cell systems for detecting neurotoxicity. Toxicology in Vitro, 2006. 20(8): p. 1569-1581.
45. Ahmadi, F. A., et al., The pesticide rotenone induces caspase-3-mediated apoptosis in ventral mesencephalic dopaminergic neurons. Journal of Neurochemistry, 2003. 87(4): p. 914-921.
46. Manoli, T., et al., Correlation analysis of histomorphometry and motor neurography in the median nerve rat model. Eplasty, 2014. 14: p. e17.

### Example 2

MEA Design Fabricated on Solid Substrates

In parallel with the activities outlined above, we are also working on a custom MEA design to be fabricated on more conventional solid substrates. The purpose is to determine specifically to what degree the permeable substrate may affect not only cell viability but also the electrophysiological responses of the cells. As shown in FIG. 5, we have finalized a design for these devices, which are easier to fabricate because they make use of more conventional microfabrication techniques. We will use these devices in parallel with the permeable-substrate MEAs in order to parse whether any deviations from previously-observed electrophysiological outcomes are due to the use of planar microelectrodes or else due to the permeable substrates.

### Example 3

Fabrication and Characterization of 3D Printed, 3D Microelectrode Arrays for Peripheral Nerve-On-A-Chip®

Abstract: We present a non-traditional fabrication technique for the realization of three-dimensional (3D) microelectrode arrays (MEAs) capable of stimulating and recording electrophysiological activity from 3D cellular networks in vitro. The technology uses cost effective makerspace microfabrication techniques to fabricate the 3D MEAs with 3D printed base structures, and metallization of the microtowers and conductive traces performed by stencil mask evaporation techniques. A biocompatible lamination layer insulates the traces for realization of 3D microtower MEAs. The process was extended to realize smaller micro-porous Platinum electrodes (30 μm×30 μm) at a height of 400 μm atop the 3D microtower using laser micromachining of an additional silicon dioxide (SiO2) insulation layer and electroless plating. A 3D microengineered, Nerve-On-A-Chip® in vitro model for recording and stimulating electrical activity of Dorsal Root Ganglion (DRG) cells has further been integrated with the 3D MEA. The 3D MEA was evaluated for electrical, electrochemical, chemical, and biological performance metrics. A decrease in impedance from 1.81 kΩ to 670Ω for the micro-tower electrodes and 55 kΩ to 39 kΩ for the 30 μm² electrodes is observed for an electrophysiologically relevant frequency of 1 kHz upon platinum electroless plating. Additionally, the capacitance increases to 3.0 mF from 0.3 mF after electroless plating which represents a 10× increase in performance. Biocompatibility assays on the components of the system resulted in a large range (˜3-70% live cells), depending on the components. FTIR analysis of the resin led to the discovery of cytotoxic compounds: TPO (diphenyl (2,4,6-trimethylbenzoyl) phosphine oxide) and nitrobenzene explaining some of this variation. Further in vitro stress tests led to the conclusion that chip thicknesses in excess of 2.5 mm gave warpage-free performance for up to 30 DIV. Lastly, a pathway to high-density 3D microelectrodes with micro-LED DLP printing was explored in this work. The fabricated 3D MEAs are rapidly produced with minimal usage of a cleanroom and are fully functional with the integrated “Nerve-On-A-Chip®” model to support the electrical interrogation of the 3D organ model for high throughput pharmaceutical screening and toxicity testing of compounds in vitro.

Introduction

The pharmaceutical industry is all too aware of the mounting costs necessary to bring a new drug to market. The average new drug requires nearly $2.6 billion and up to 15 years to obtain market approval, as well as an additional $312 million for post-approval research and development to maintain approval [1]. Unfortunately, there is a poor track record of drug development in conventional preclinical models leading to successful clinical therapeutics. For neurological applications in particular, it is estimated that as high as 92% of neurological drugs that enter Phase I clinical trials will never be marketed to consumers due either to unacceptable toxicity or lack of efficacy in humans [2]. Clearly, current preclinical models including both animal and in vitro models have very limited predictivity when it comes to the translation of preclinical success to clinical trials. Animal models may provide relevant in vivo information, but they are time-consuming and labor intensive (low throughput), while on the other hand, higher throughput in vitro systems are typically restricted to basic neural cultures consisting of randomly growing dissociated cells in two dimensions and incapable of providing relevant in vivo information. Thus, higher throughput systems capable of providing relevant in vivo metrics are highly desired, leading to the development of advanced microphysiological systems (MPSs) or “organs-on-chips,”[3, 4].

For the nervous system, where electrophysiological and histological evaluation are the gold standard measurements to evaluate neuropathies [5], a biomimetic in vitro system capable of providing clinically-relevant metrics such as nerve conduction velocity and nerve fiber density is expected to improve clinical predictivity. Previously, we developed a novel biomimetic in vitro Nerve-On-A-Chip® (NOaC) system using either animal [6 also Huval (46)] or human cells [7], where axons can be extracellularly stimulated in a 3D polarized structure resulting in unidirectional propagation of signal and thus, evaluation of compound action potentials (CAPs). While these innovative systems provide in vivo information in an in vitro setting, electrophysiological testing included labor-intensive manual placement of stimulating and recording electrodes using micromanipulators which hampered the rate of testing compared to other higher throughput 2D multi-electrode array (MEA) systems.

To overcome this challenge, we are planning to integrate our NOaC system with 3D microelectrodes to automate the process of stimulation and recording and hence, increase the throughput of the system making it amenable for screening therapeutic compounds on a large scale. 3D electrodes are expected to interrogate a larger number of diverse axonal fibers to realize population-based electrophysiological responses more akin to in vivo nerve tissue, as compared to other 2D MEA platforms [8, 9] previously developed for the evaluation of nerve conduction velocity. Additionally, the planar configuration of conventional MEAs makes them inadequate to capture signals that occur at a certain height when cultures mature to obtain a 3D form [10, 11]. The capture and analysis of signals from thicker, mature tissues is especially important in neurological models on a chip [12]. The goal of this paper is to define a microelectrode design that is integrated into the unique 3D hydrogel environment for much more rapid electrophysiological testing.

Conventional 2D MEA fabrication typically involves lithography, metallization, and etching techniques on silicon or glass substrates [13, 14]. Since lithographic techniques on non-planar surfaces is particularly challenging, monolithic 3D MEA fabrication techniques are rare. Recently, there have been tremendous efforts invested into the development of a variety of 3D cell culture systems and as a result, there is a growing need to extend in vitro MEAs to the third dimension [15-21]. 3D MEAs would allow for simple, rapid screening and measurement of network dynamics for the study of 3D microengineered systems for central or peripheral nervous system applications.

3D MEAs have been fabricated on traditional substrates such as silicon and glass as well as nontraditional substrates such as parylene, SU-8, various metals, polyimides, etc. [22]. Silicon based 3D MEAs such as Michigan probes [23-25], Utah Array [26-29] and European NeuroProbes [13, 30-35] are at the forefront of 3D MEA development for in vivo applications. Additionally, metal [36], glass [37] and polymer probes as 3D MEAs have also been investigated including 3D MEAs fabricated from technologies such as Electrical Discharge Machining (EDM) [38], polyimide or Kapton [39, 40] micromachined, Parylene [41, 42] based, SU-8 [10] based active 3D microscaffold technology with microelectrode and microfluidic functionalities and Metal Transfer Micromolding (MTM) [11]. However, fabrication of most of the aforementioned types of 3D MEAs require extensive processing in the cleanroom and/or involve complex fabrication/assembly methodologies making them expensive and available only to end users with extensive facilities not to mention the time spent from a concept to a final device [Table S1].

For cost-effective and “on demand” manufacturing processes for 3D MEA fabrication, introduction of rapid prototyping technologies utilizing robust, benchtop based, design-to-device strategies is the logical next step. In fact microfabrication technologies for nanobiosensors, biomedical micro-electro-mechanical systems (BioMEMS) and micro-total analysis systems (MicroTAS) applications have been transitioning away from lithographic techniques towards non-traditional benchtop based fabrication processes as most biological devices do not require the sophistication of the cleanroom environment [43]. A makerspace provides easy access to a variety of tools in an intimidation-free environment to application developers while providing immense flexibility in varied materials and allowing for rapid design changes with scalable fabrication and assembly. We have recently introduced the concept of ‘Makerspace Microfabrication’ [44] which was used for the realization of biological microdevices such as 2D Microelectrode arrays (MEAs), microneedles (MNs) and Microfluidic channels (MFCs). Our ‘Makerspace Microfabrication’ utilizes traditional technologies as needed and has been extended to include new toolbox technologies such as 3D spin cast insulation and electrospinning [45].

In this paper, we report the first application of ‘Makerspace Microfabrication’ to realize 3D MEAs for electrophysiological assessment of a 3D microengineered system. The process flow for the device begins with 3D printing to realize the physical structure of the microtowers. The 3D microtower MEAs have a base diameter of 250 μm and a height of 400μm. Two patches, each containing ten recording sites in the form of 3D microtowers were designed. The arrangement of the ten microtowers were such that they would match with the geometry of the 3D microengineered Nerve-On-A-Chip®[46] which comprises a circular region (ganglion) leading into a straight channel (neural tract). The microtowers would overlap both with the circular ganglion and the neural tract to act as recording/stimulating electrodes. A metallization layer, realized by stencil mask evaporation techniques, defines the metallized towers and conductive traces. A biocompatible lamination layer is used to insulate the traces thereby enabling realization of 3D microtower MEAs onto which the 3D dual hydrogel constructs for incorporation of dorsal root ganglia (DRG) explants were defined. An additional e-beam evaporated SiO2 layer defines a “fine” insulation for the 3D MEA. The metallization and SiO2 evaporation atop 3D printed substrates demonstrate the collaboration between non-traditional and semiconductor processing technologies, a cornerstone of ‘Makerspace Microfabrication’. The hierarchical nature of the process also allows for subtractive manufacturing techniques such as micromilling and laser micromachining to define the insulation layer. Such a buildup allows for functionalities to be added by every process to realize complex designs. Optical and SEM imaging have been performed to characterize the various constituent processes. Full spectrum impedance analysis of the fabricated electrodes confirms microelectrode nature whose capacitive behavior can be further enhanced by electroless deposition of platinum. Both microtower electrodes and smaller 30 μm² are further demonstrated along with chemical and biological characterization of the MEA materials.

Materials and Methods

The device fabrication, characterization, and assay methodologies are described in detail in this section.

3D Printing

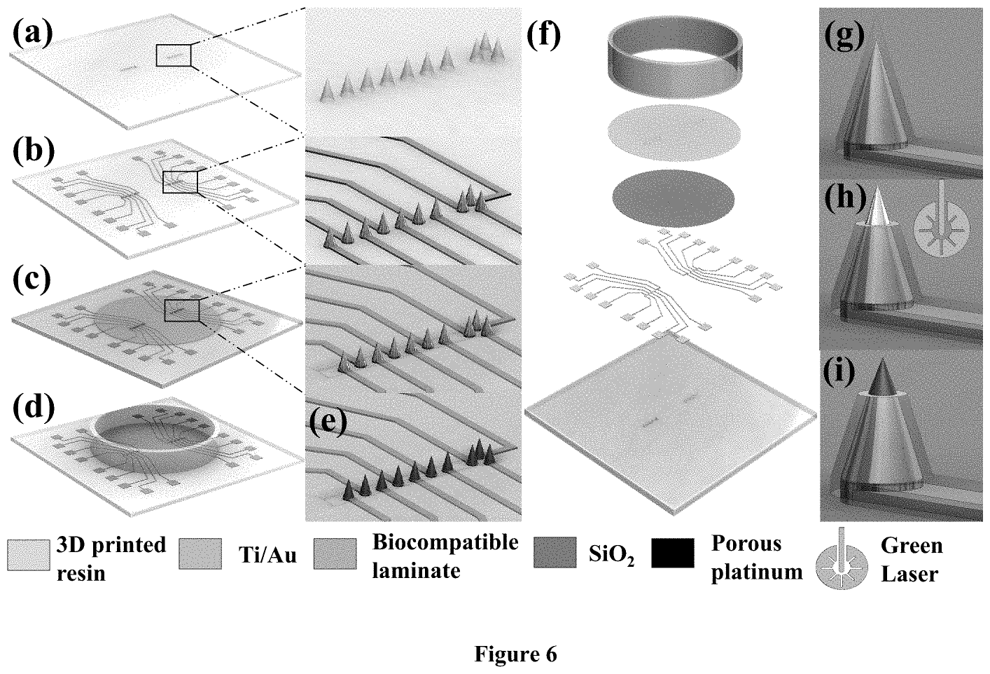

The 3D MEAs were designed in Solidworks (2016 x64 bit edition, Dassault Systems Inc., Waltham, Mass., USA). The MEA chip has a size of 49 mm×49 mm×2.5 mm to ensure connectivity with the Multi-Channel Systems (Reutlingen, Aspenhaustrasse, Germany) recording amplifiers. Two patches, each containing ten recording sites in the form of 3D towers were designed. The microtowers had a base diameter of 250 μm and a height of 400 μm. Seven microtowers having a pitch of 600 μm were placed along straight line while three microtowers were placed in a centrosymmetric fashion along the same straight line at a distance of 750 μm from the linearly placed electrodes. FIG. 6(a) shows the schematic of the 3D printed geometry with an exploded view of one of the microtower patches containing ten recording/stimulating sites. The designed CAD file was directly printed in a 3D SLA printer (Form Labs Form 2, Somerville, Mass., USA) with a laser wavelength of 405 nm using a photopolymer clear resin (FLGPCL04, Formlabs, Somerville, Mass., USA). The device was printed at an angle of 45° with the horizontal which has been found to be optimum for such 3D geometries[44]. Upon completion of the 3D printing, the devices were removed from the build platform and rinsed in an isopropyl alcohol (IPA) bath with mild agitation for 10 minutes. The rinse cycle was repeated for a second time in a fresh IPA bath. The device was subsequently dried in nitrogen followed by the removal of the support structures. For acetone vapor polishing, the fabricated devices were placed on top of an aluminum foil that was placed inside a 1-liter glass beaker. Kimwipes (Kimtech, Roswell, Ga., USA) were soaked in acetone and hung from the interior edges of the beaker. The beaker was sealed with Parafilm®, (Sigma-Aldrich) and the 3D printed device were polished in acetone vapor for 4 minutes.

Metallization

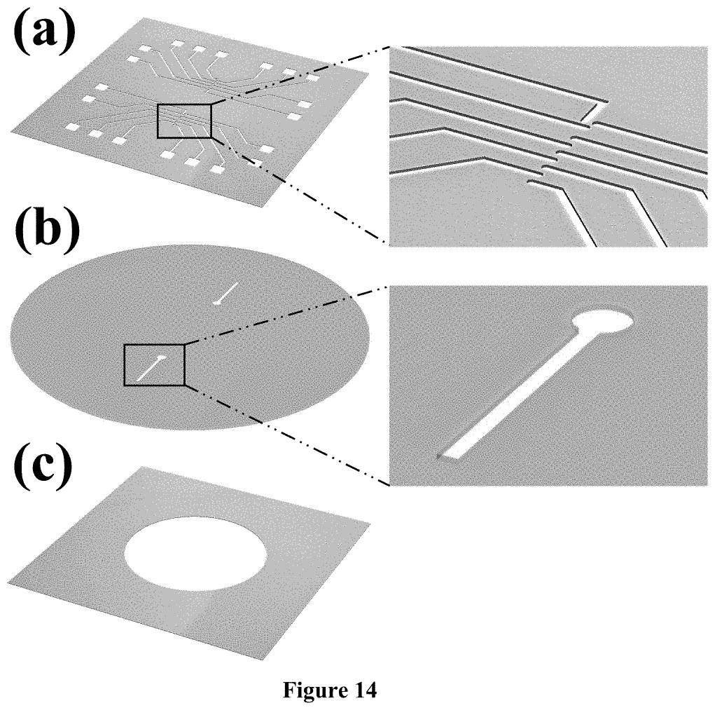

Electron beam evaporation of Ti/Au was performed through a micromilled stainless steel stencil mask for metallization of the 3D microtowers and definition of the conducting traces (200 μm wide) terminated by package landing pads (2.2 mm×2.2 mm). For the fabrication of the stainless-steel mask a 90-degree T-8 Mill Tool (150 μm-250 μm diameter; T-Tech, Peachtree Corners, Ga., USA) was spun at 55,000 rpm with a feed rate of 2 mm/sec in a T-Tech J5 Quick Circuit Prototyping Systems to micromill the stainless-steel sheet (80 μm thick; Trinity Brand Industries, Countryside, Ill., USA). The 3D printed device and the micromilled mask were aligned under a stereoscope and a metallization layer comprising titanium and gold (Ti, 4N5 purity pellets and Au, 5N purity pellets, both from Kurt J. Lesker, Jefferson Hills, Pa., USA) was deposited by electron-beam (E-beam) evaporation (Thermionics Laboratory Inc., Hayward, Calif., USA). The Ti and Au layers were deposited in a vacuum of 3.1×10-6 Torr to a thickness of 10 nm at a deposition rate of 1.0 nm/s and 100 nm at 1.0 nm/s, respectively. FIG. 6(b) shows a schematic of the metallization pattern with an exploded view of one of the recording/stimulating patches. The schematic of the shadow mask is shown in supplementary information [FIG. 14 (a)].

Lamination

A biocompatible laminate layer (Medco®RTS3851-17 adhesives ˜50 μm thick underneath a poly ethylene terephthalate (PET) ˜20 μm thick; Medco Coated Products, Cleveland, Ohio, USA) is subsequently bonded to the 3D printed chip to insulate the traces thereby enabling realization of 3D microtower MEAs with electrodes having a size of the entire 3D printed structure. The biocompatible laminate is micromilled prior to its alignment and attachment to have openings corresponding to the size of the two patches of 3D tower arrays, each containing ten recording sites. The openings in the biocompatible laminate layer correspond to the Nerve-On-A-Chip® dimensions which comprises a circular region (˜800 μm in diameter) leading to a straight channel (4.2 mm long and 500 μm wide). The diameter of the biolaminate layer was 32 mm, which is marginally greater than the diameter of the culture well to be affixed later onto the device. The micromilling was performed using the T-8 Mill Tool which was spun at 45,000 rpm with a feed rate of 5 mm/sec. FIG. 6(c) shows the schematic of the lamination process with an exploded view of one of the recording/stimulating patches. The schematic of the micromilled lamination along with its geometry is depicted in supplementary information [FIG. 14 (b)].

Packaging

A culture well having an outer diameter of 30 mm and a thickness of 2.1 mm is 3D printed, coated with PDMS to enhance biocompatibility and bonded using a biocompatible epoxy (Epo-Tek® 353ND) to realize the final device. The height of the culture well is 3 mm. Parts A and B of the epoxy were mixed in ratio of 10:1 (by weight) and affixed onto the 3D microtower device as depicted in FIG. 6(d). The packaged device was cured at 40° C. for 4 hours. The devices were tested for leaks with a drop of IPA and DI water prior to the electroless platinum plating and electrical, electrochemical, and biological characterizations.

Electroless Platinum Plating

For electroless deposition of micro-porous platinum (commonly known as platinum black) on the gold coated 3D microtower MEAs, 0.01% wt. platinum solution was prepared using 3.75 mL (˜8% chloroplatinic acid from Sigma-Aldrich), 0.2 mL of 0.005% wt. lead acetate (Sigma-Aldrich), 4.065 mL of 1.23M HCl (Sigma-Aldrich) and 2.085 mL of DI water. Approximately 5 mL of this solution was transferred to the MEA culture well and passive electroless plating was performed for 6 hours for obtaining platinum coverage on the microtower electrodes. The completed device was subsequently rinsed with DI water and dried with nitrogen. FIG. 6(e) depicts a schematic of the individual electrodes of different sizes after the electroless plating of micro-porous platinum.

Insulation and Laser Micromachining of Microelectrodes

To realize smaller electrodes, an insulation layer of SiO2 is defined atop of the 3D microelectrode towers after Ti/Au metallization described in Section 2.2. A manually rotated e-beam evaporation of SiO2 pellets (4N5 purity from Kurt J. Lesker, Jefferson Hills, Pa., USA) was performed. The deposition was performed through a micromilled stainless steel stencil mask as depicted in supplementary information [FIG. 14(c)]. The deposition rate was 10 nm/s with a target SiO2 thickness of 400 nm. This was followed by the lamination (Section 2.3) and packaging (Section 2.4) of the device. One may note here that the biocompatible laminate layer is not required for the SiO2 insulated 3D MEAs. However, as the cut-out of the laminate layer [FIG. 14 (b)] is similar to that of the Nerve-On-A-Chip® design[46] it additionally may serve to contain the microengineered 3D neural culture. FIG. 6(f) shows the exploded view of the fabricated device with an evaporated layer of SiO2. FIG. 6(g) shows the close-up of a singular microtower with SiO2 insulation. The uniform SiO2 insulation layer can subsequently be selectively laser micromachined to define microelectrodes of a size similar to commercial MEAs as depicted in the schematic in FIG. 6(h). These 3D microelectrodes 30 μm² in size and were realized using laser micromachining (a 4 ns laser pulse at 532 nm having an energy level of 1.2 mJ) utilizing QuickLaze 50ST2 (Eolite Lasers, Portland, Oreg., USA). Platinum electroless plating of the laser micromachined electrodes can be subsequently carried out as outlined earlier in Section 2.5 and is depicted schematically in FIG. 6(i).

Imaging, Chemical, Electrical and Stress Measurements

Optical and Scanning Electron Microscopy (SEM) images were performed at all stage of the microfabrication development using BX51M microscope (Olympus, Center Valley, Pa., USA) and JSM 6480 (JEOL, Peabody, Mass., USA) respectively.

Fourier-Transform infrared spectroscopy was performed for the 3D printed resin (FLGPCL04, Formlabs, Somerville, Mass., USA) to access the chemical composition along with the various functional groups present in the material which would impact its suitability for long term in vitro cultures. FTIR measurements were conducted using a PerkinElmer Spectrum 100 FT-IR Spectrometer (Waltham, Mass., USA) where 1-5 mg of sample was used for each FTIR trial.

For stress testing of the devices in culture-like conditions, test devices (N=5) were fabricated in thickness intervals of 0.5 mm, from 1 mm to 3 mm. The devices were submerged in a petri dish containing 0.025× Dulbecco's Phosphate Buffer Solution (Thermo Fisher Scientific, Waltham, Mass., USA) to imitate both hydration and cell culture media, simulating the culturing conditions. The devices were placed in a cell culturing incubator (NUAIRE, NU-5100 Series 2, MN, USA) at 37° C., 90% RH and 12% CO2 for 30 days in vitro (DIV). Warpage data was obtained twice daily for 30 days, utilizing feeler gauge (0.02-1 mm Thickness Gap Metric Filler Feeler Gauge, Jinghua Company, China), which allowed for the warpage from the base of the device to be measured on a flat surface. A small counter weight (e.g. a glass slide) was placed on top of the culture well to hold it in place, and the feeler gauge was inserted under the base to identify the thickness of the impending curvature. This value was recorded for all four sides of the device, followed by data averaging across devices during daily measurements. Phosphate buffered saline (PBS) was added at the beginning of each day to account for evaporation.

Impedance measurements of the MEAs were performed with both the microtower and the microelectrode 3D MEAs using a Bode 100 Impedance Analyzer (Omicron Labs, Houston, Tex., USA) with Dulbecco's Phosphate Buffer Solution as the electrolyte. The impedance scans were carried out from 10 Hz to 1 MHz with a platinum wire (eDAQ, Denistone East, Australia) as the counter electrode. Cyclic voltammetry (CV) was performed using a Potentiostat 466 system (from eDAQ) and a three-electrode setup with a silver/silver chloride (Ag/AgCl) wire acting as the reference electrode and a Pt. wire used as the counter electrode. PBS was used as the electrolyte. To estimate the capacitance of the electrodes CV scans were performed from −1V to 1V with scan rates of 20 mV/s, 40 mV/s, 60 mV/s, 160 mV/s and 250 mV/s.

Nerve-On-A-Chip® Fabrication and Integration with 3D MEA

A dual-hydrogel scaffold was fabricated on semi-permeable membranes (Transwell® insert 0.4 μm pore/PES; Corning) using photolithography. The cell-impermeable outer hydrogel mold with an open keyhole center was created using a solution of polyethylene glycol dimethacrylate (PEG 1000, Polysciences) and photo-crosslinked with lithium phenyl-2,4,6-trimethylbenzoylphosphinate (LAP, Sigma Aldrich). The outer hydrogel is fabricated such that the 3D electrodes are exposed within the central keyhole area as depicted in FIG. 14 (b). 10% w/v PEG and 1.1 mM LAP solutions were mixed as a 1:1 solution then sterile-filtered with a 0.22 μm filter. 0.6 ml of the solution was added to the Transwell® insert and positioned under the lens of a Digital Micromirror Device (DMD, PRO4500; Wintech Digital Systems Technology Corp). The mask and polymerization parameters were created in the DMD software. The solution was irradiated for 30 seconds using ultraviolet light at 385 nm wavelength. After treatment, excess PEGDMA/LAP was washed using 2% Antibiotic-Antimycotic PBS solution three times on the top and bottom of the inserts for 10 minutes. Wash buffer was removed from the insert and the inner keyhole-shaped channel.

MEAs were prepared for cell culture by first sterilizing with UV for 20 minutes under a cell culture hood. Samples were washed three times, each for 8 minutes consisting of phosphate buffered saline (PBS) pH 7.4 without calcium and magnesium and with 1% Antibiotic-Antimycotic (100×, Gibco, ThermoFisher). Samples were then dried under a cell culture hood. The keyhole void containing the 3D microelectrodes was encased in 10 μl of 8% Matrigel Basement Membrane Matrix (Corning) hydrogel, then placed into an incubator for 15 minutes to solidify.

Primary nerve tissue used for experiments were extracted following animal handling and tissue harvesting procedures in accordance to the Institutional Animal Care and Use Committee (IACUC) in part with Tulane University. DRG consisting of peripheral sensory neurons and glial Schwann cells were isolated from Long-Evans rat, embryonic day 15 pups (Charles River, Wilmington, Mass.). DRGs were then directly placed into the Matrigel on the MEA. The cells were cultured in 20 ml of media consisting of Basal Eagles Medium (Thermofisher), 15% fetal bovine serum (HyClone), Insulin Transferrin Selenium (ITS, Thermofisher), Glutamax (Thermofisher), Antibiotic-Antimycotic, 4 g/L D-glucose (Sigma), 10 ng/ml of Nerve Growth Factor (NGF) (R&D systems), and 50 μg/ml of L-ascorbic acid (Sigma). Cultures were kept at 37° C. with 5% CO2 incubator.

Nerve-On-A-Chip® Measurements

The DRGs were analyzed for biocompatibility and viability after 10 days of incubation. The MEAs were washed three times with PBS then incubated with 3 μM propidium iodide (PI) (dead cells staining) (Invitrogen) and 4 mM calcein AM (live cells staining) (Biotium) for 30 mins at 37° C. The samples were then washed three more times with PBS, and imaged using a fluorescent microscope. Images were analyzed by counting total number of stained cells using ImageJ (NIH) Fiji software package. Briefly, for each image, background fluorescence was removed through software thresholding and maximum intensity of the stained cells were counted using point selectivity. The number of cells found for each sample was averaged (n=4 DRGs per sample). A one-way analysis of variance (ANOVA), was calculated for live cell counts only (p>0.05). Unless otherwise specified, data are presented as mean (±) standard deviation.

Results and Discussions

In this section we detail the results from the microfabrication process development of the 3D MEA. The electrical and electrochemical results of the 3D microtower MEAs as well as 30 μm² 3 D MEA are discussed. Additionally, we will describe integration, biocompatibility, in vitro stress testing, and chemical analysis when coupling the device with the Nerve-On-A-Chip® model [46].

Device Microfabrication

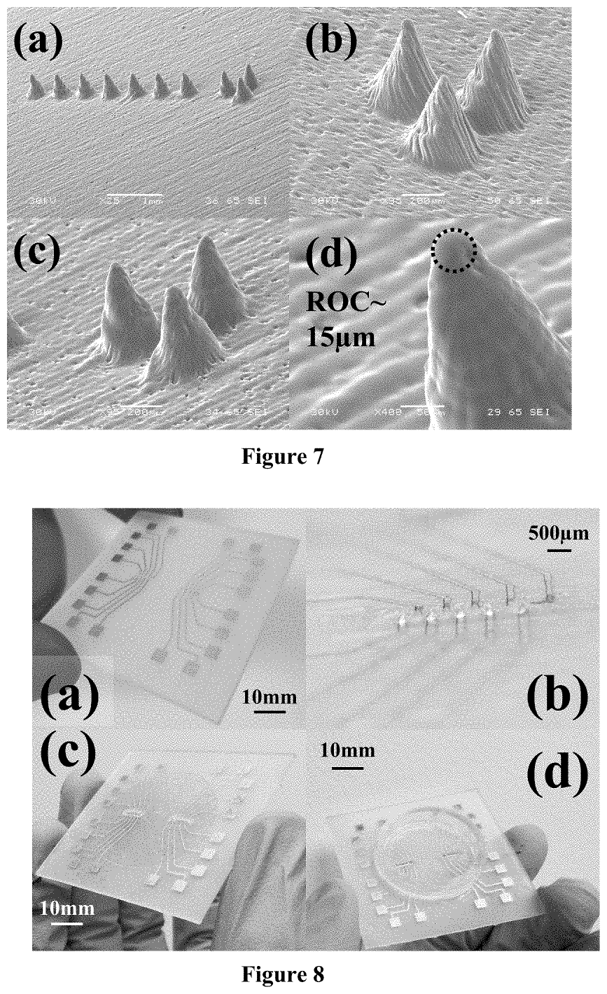

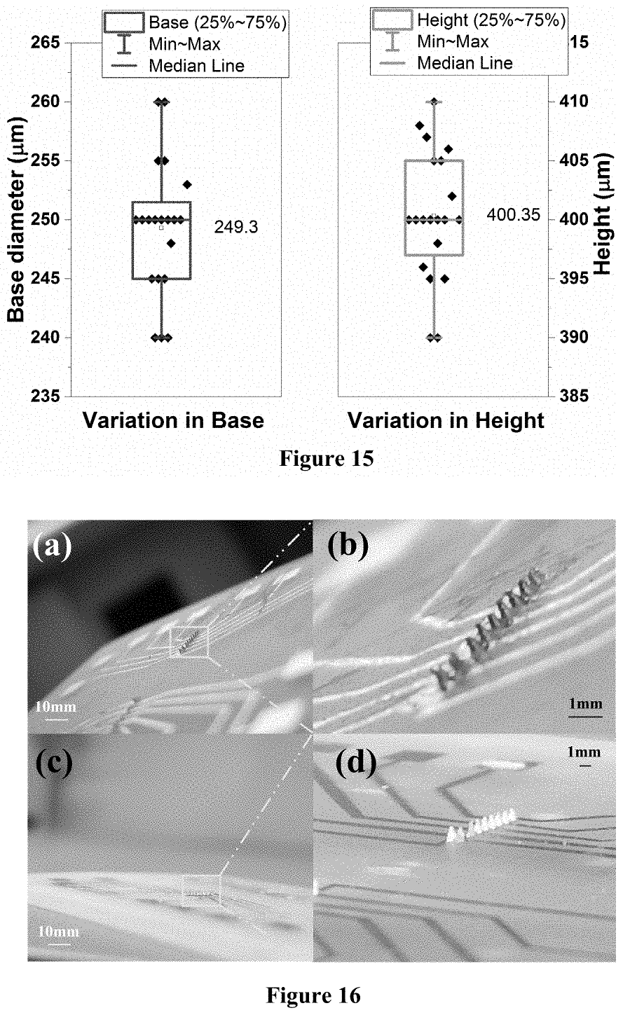

FIG. 7(a) shows the SEM image of the 3D printed 3D microtowers in one patch of the recording/stimulating MEA. It is observed that the microtower dimension closely matches the design dimension of 250 μm base width and 400 μm height. A box plot of N=20 electrodes showing variation in base diameter and height is shown in FIG. 15. Such an arrangement of the microtower MEAs allows for the recording and stimulating sites to be well-matched with the geometry of the 3D microengineered Nerve-On-A-Chip® [46]. The three microtowers are arranged in a centrosymmetric fashion that is designed to come into contact with a spherical bulb of neural ganglion of the Nerve-On-A-Chip® and would act as individual recording/stimulation sites while the seven microtowers would overlap with the neural axon tract and act as recording/stimulating electrodes. FIG. 7(b) shows the close-up view of the 3D microtowers in the circular region and it is observed that the microtower geometry has striations inherent of SLA based 3D printing. Such striations originate when each of the 3D printed layers are covalently stitched to the subsequent layer. Acetone vapor polishing can be employed to isotropically etch the outer surface of the 3D microtowers to reduce the striations as seen in FIG. 7(c). However, the striations could be a useful method to increase the surface area of the electrodes. The isotropic etch process results in a microtower tip having a radius of curvature (ROC) of ˜15 μm as depicted in FIG. 7(d).

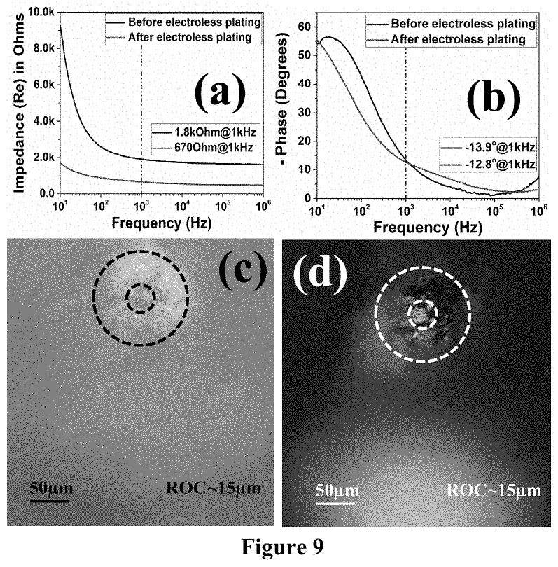

FIG. 8(a) shows the photomicrograph of the device after deposition of Ti/Au to obtain the metallized 3D microtowers and conducting traces. FIG. 8(b) shows a close-up view of the ten metallized microtowers corresponding to a single recording/stimulating patch. FIG. 8(c) shows the selective lamination of the device to insulate the traces and thereby realize the 3D microtower MEAs after attachment of the 3D printed culture well as seen in FIG. 8(d). At this juncture, the device is ready for electroless platinum plating, electrical, chemical and electrochemical measurements and integration with the 3D microengineered Nerve-On-A-Chip®.

Electrical and Electrochemical Characterization of the 3D MEAs

Electroless plating of micro-porous platinum results in a coating that is extremely resistant to chemical corrosion, biocompatible, and has reduced electrical impedance for recordings [18]. Additionally, the layer's low threshold potential makes it interesting for applications in electrical stimulation [47]. FIGS. 9(a) and 9(b) depict the full spectrum impedance and phase response of the 3D microtower MEAs before and after electroless plating of micro-porous platinum (average of N=10 of each electrode type). It is observed that the magnitude of impedance decreases upon electroless deposition of porous platinum which can be attributed to the increased surface roughness of the 3D MEA which leads to an increase in the surface area of the electrode. A decrease in impedance from 1.81 kΩ to 670Ω is observed for an electrophysiologically relevant frequency of 1 kHz. The phase spectrum is observed to shift from −60° to near 0° which implies that the overall characteristics of the electrode-electrolyte interface is governed by the double layer capacitance (CDL) at low frequencies and becomes more resistive at higher frequencies as the solution resistance of the electrolyte begins to dominate the electrode-electrolyte interfacial impedance as it has been observed with other MEAs [48]. At 1 kHz, a phase of −13.9° and −12.8° is observed the 3D microtower MEAs before and after electroless plating respectively. It is interesting to note the capacitive behavior of the 3D tower MEAs from the phase response at frequencies as low as 10 Hz which implies large CDL values. However, at very low frequencies (<10 Hz), the impedance related to double layer capacitance is large enough to be omitted and can be replaced by an open circuit and a trend towards more resistive behavior is seen from the phase response of the 3D microtower MEAs before electroless plating [48].

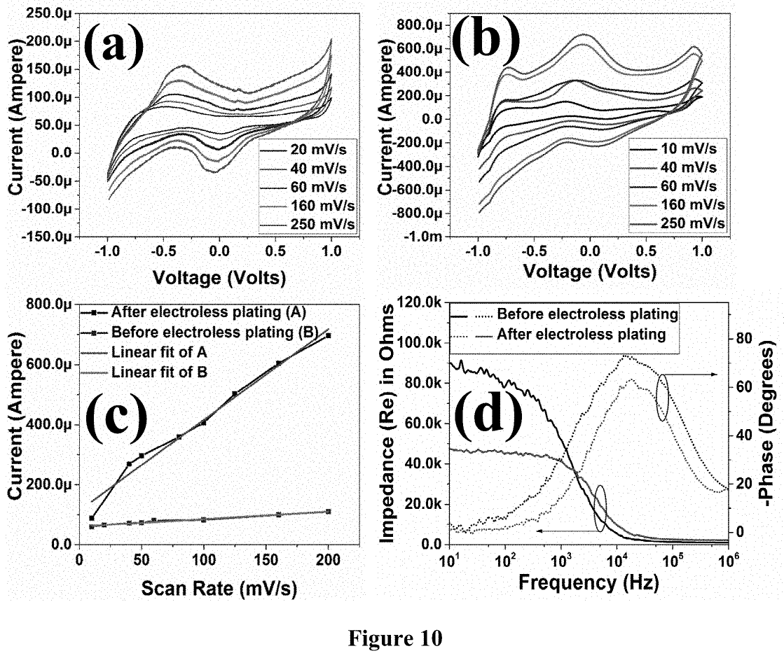

Optically, micro-porous platinum is observed from the gold surface turning black as evident from FIGS. 9(c) and 9(d). A photomicrograph of ten micro-porous platinum electrodes of a single patch is provided in supplementary information [FIGS. 16 (a) and (b)]. The photomicrograph of the 3D microtower MEAs prior to electroless plating is also provided [FIGS. 16(c) and (d)] for easy visual referencing. Scan rate variations during cyclic voltammetry of the 3D tower MEAs have been performed to estimate the change in double layer capacitance after electroless platinum plating. FIGS. 10(a) and 10(b) depict the scan rate variations of the 3D tower MEAs before and after electroless plating. A linear fit of the current vs. scan rate plot has been performed with the scan rate variation data and the capacitance values were extracted from the slope of the graphs [44, 49]. The capacitance increases to 3.0 mF from 0.3 mF after electroless plating which is an order of magnitude (10×) increase in capacitance as evident from the slope of the graph shown in FIG. 10(c) demonstrating the power of micro-porous platinum in the control of the surface texture of the MEAs and hence demonstrating improved abilities to capture small neurological signals. For the smaller electrodes (30 μm² in size), the reduction in the electrode size results in a significant increase in the impedance and consequently decreases the signal-to-noise ratio (SNR) [18]. As a result, we deposited micro-porous platinum on these electrodes, for improved SNR. FIG. 10(d) shows the full spectrum impedance and phase response of the 30 μm² microelectrodes before and after electroless plating of platinum. A significant decrease in impedance (N=2) from 55 kΩ to 39 kΩ is observed for the electrophysiologically relevant frequency of 1 kHz. This value is very much in range for nano-porous platinum 2D electrodes of a similar size that are commercially available[50]. The phase signature of the smaller electrodes is also shown in the same figure and it is observed that the smaller size of the electrodes results in a lower value of CDL which manifests as a resistive behavior of the MEAs for frequencies up to 100 Hz. As the frequency increases the effect of CDL becomes more pronounced and the electrode-electrolyte interfacial impedance becomes more capacitive [49].

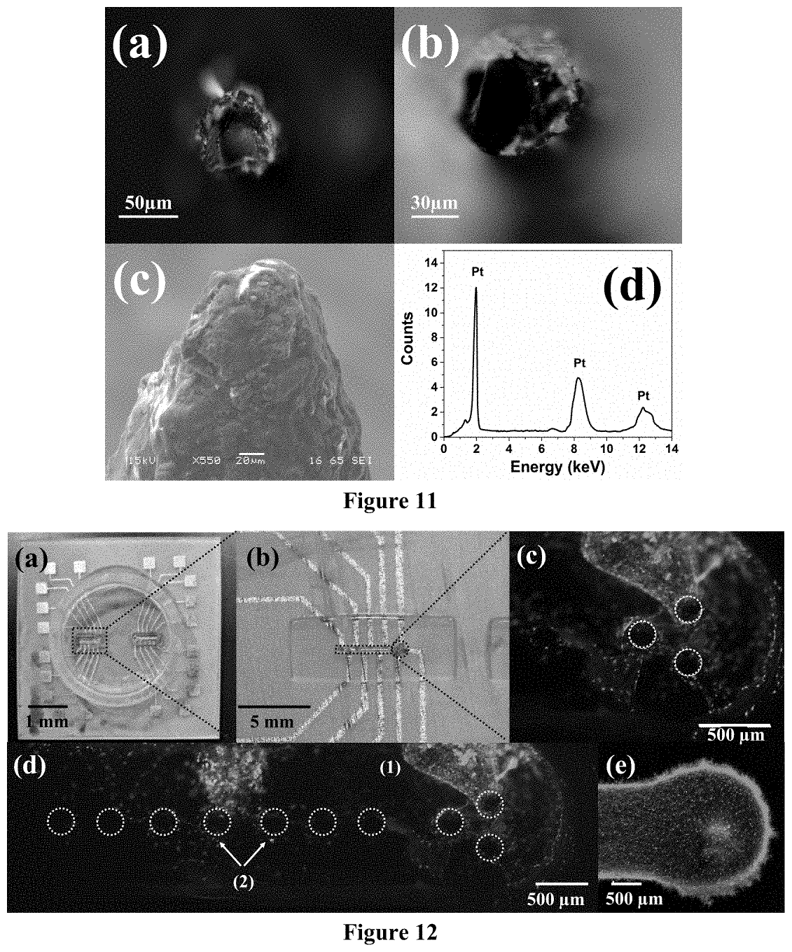

FIG. 11(a) shows the close-up microphotograph of the tip of 3D microtower after SiO2 deposition. The interference of light due to the transparent nature of SiO2 imparts a distinct blue-violet color to the microelectrode. FIG. 11(b) depicts the distinctive black color of the micro-porous platinum on the top of the microtower after electroless plating on the laser micromachined recording site. FIG. 11(c) shows the SEM image of the tip of the micro-porous platinum electrode with significant roughening due to micro-islands of platinum upon electroless plating. The effect of this phenomenon is a larger surface area and a lower value of impedance. In order to validate the presence of platinum, Electron Dispersive Spectroscopy (EDS) analysis of the MEAs were performed. FIG. 11(d) confirms the presence of platinum to almost 90% wt. after electroless deposition on the microporous islands formed on the tip of the 3D microtower due to peaks attributed to platinum[51].

Biological and Chemical Characterization of 3D MEAs

The aim of the biocompatibility study was to devise a simple and rapid method to evaluate cell survival within the 3D MEA samples. Calcein AM is a widely used stain that can be introduced into cells via incubation. Once inside the cells, calcein AM is hydrolyzed by endogenous esterase into a green fluorescent molecule retained in the cytoplasm. Propidium iodide (PI) is a popular red-fluorescent nucleic acid counterstain that is impermeable to intact membranes of live cells. FIG. 12(a) shows the DRGs on the 3D microtower MEAs. One recording patch containing ten recording/stimulating sites is marked in blue. The close-up of one of the patches containing ten recording/stimulating sites is shown in FIG. 12(b). The keyhole filled with Matrigel® Matrix is marked in blue and the PEG construct is marked in red. Composite images of live (green) and dead (red) cells of a DRG grown on top of the MEA surface in the circular portion of the Nerve-On-A-Chip® is shown in FIG. 12(c). FIG. 12(d) depicts the stitched composite image demonstrating DRG placed onto the MEA for a patch containing ten recording/stimulating sites. It is clearly seen that neural cells are wrapped around the 3D microtowers suggesting anchoring of the construct. FIG. 12(e) shows the close-up view of the circular region of the Nerve-On-A-Chip® for a control sample.

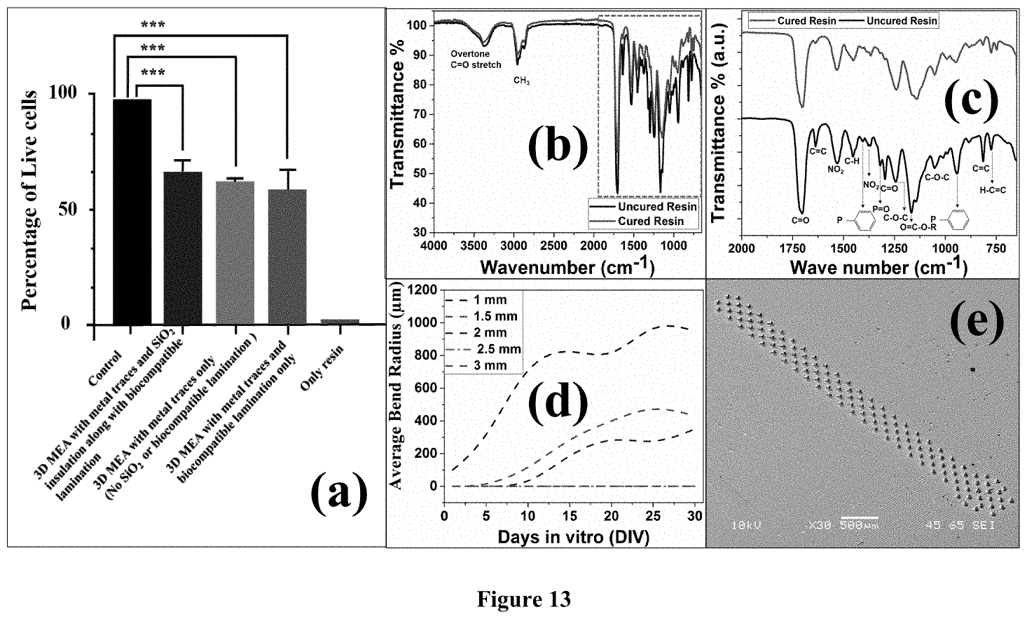

The quantification of the biocompatibility of the samples and the control is depicted as percentage of live cells [FIG. 13(a)]. It is observed that the control sample shows the highest percentage of live cells (˜96%) in contrast to the photopolymer clear resin used for 3D printing the substrate of the 3D MEA (˜3%). The other test beds in the 3D MEA material set are in between the two extremes (˜55-70%) suggesting potential cytotoxic leachants from the 3D printing resin material. In order to investigate this potential cytotoxicity, FTIR analysis of the 3D printed material was performed. FIG. 8 (b) shows the FTIR results for the uncured as well as the cured resin. An exploded view of the fingerprint region of the FTIR (2000 cm-1-650 cm-1) is additionally shown in FIG. 8 (c). The analysis reveals that the uncured base monomer/oligomer is a methacrylic acid ester validated by the C═O stretch at 1700 cm-1, C—O—C (methacrylate) asymmetric stretch at 1250 cm-1 and C—O—C (methacrylate) symmetric stretch at 1050 cm-1. This conclusion is consolidated by the oscillation of the ester group (O═C—O—R) at 1168 cm-1. It is important to note that the signals corresponding to the C═C acrylate moiety at 1638 cm-1 and 816 cm-1 significantly decreased after curing, indicating the consumption of double bonds due to the network formation on polymerization. For the same reason, the signal representing to the oscillation of the ester group shifted from 1168 cm-1 to 1137 cm-1, signal corresponding to H—C═R at 772 cm-1 weakened and shifted and the signal at 1453 cm-1 due to C—H bending due to methylene decreased. These signals along with the overtone from the C═O stretch which manifests itself at 3364 cm-1 in the monomer/oligomer/polymer confirm that the polymer is a methacrylic acid ester which is not a hazardous compound as per Globally Harmonized System (GHS) classification [52]. Thus, the cytotoxicity of the material is not due to the base polymer but the presence of the photoinitiatiors and/or other compounds such as the thermal polymerization inhibitor. While the photoinitiator acts as catalyst for photo polymerization, thermal polymerization inhibitors are used to prevent thermal polymerization or polymerization over time to increase the shelf life of the resin [53]. Typical photoinitiators present in commercial resins may range from phosphine oxide compounds, hydroxyl-acetophenones, benzophenone compounds, camphorquinone, 1-hydroxy cyclo hexyl phenyl ketone, triarylsulfonium salt etc. [54],[55]. However, from the FTIR analysis the P═O stretch at 1320 cm-1 confirms that the photoinitiatior is indeed based on phosphine oxide. Further, the signals arising at 1406 cm-1 and 945 cm-1 from the phenyl-P bonds corroborate that the photoinitiator is either TPO (diphenyl (2,4,6-trimethylbenzoyl) phosphine oxide) or BAPO (phosphine oxide, phenyl bis(2,4,6-trimethylbenzoyl). BAPO results in a significant yellow discoloration typically observed after curing[56]. The same effect is not observed with TPO and as the resin is essentially “clear” upon photo polymerization the photoinitiator may be concluded as to be TPO.

Two other important signals additionally emerge from the fingerprint region; the absorption peaks at 1530 cm-1 (asymmetric stretch) and 1365 cm-1 (symmetric stretch) arising from the NO group attached to an aromatic ring. This suggests the presence of nitrobenzene [57], a compound which is commonly used to prevent thermal polymerization over time. Both TPO and nitrobenzene are hazardous compounds as classified by GHS [58],[59] and are most-likely the reason for the significantly reduced DRG cytocompatibility observed for the substrate (˜3%). However, as we add functionalities to the device such as metallization (biocompatible Ti/Au), coarse insulation (PET-based biolaminate layer) and biocompatible SiO2 insulation to realize 30 μm² electrodes the percentage of the live cells is observed to increase to ˜70% [FIG. 13(a)]. Evaporation of SiO2 to realize the smaller electrodes inevitably covers the inter-electrode regions exposed to the photopolymer (gold metallization covers only the 3D microtowers) which increases the biocompatibility of the device in addition to making the device capable for realization of small recording sites of ˜30 μm².

As the chemical analysis reveals, the base polymer being a methacrylic acid ester will be prone to water/media sorption during cell culturing experiments potentially leading to warpage. To evaluate the water sorption characteristics, test resin samples of different thicknesses as discussed were placed in peak physiological conditions, to best mimic the cell culturing conditions. The devices were fully submerged as to ensure that the hydration constant for the experiment was always as close to 100% as possible, and to obtain results over a reasonable time-scale. As can be seen in FIG. 13(d), the warpage of the resin-based devices was not constant over the thickness range (1-3 mm), but showed a downward trend with increasing thickness of the 3D MEA. The peaks would indicate full saturation of the devices, while small reductions in the data demonstrate a fluctuating equilibrium. These reductions occurred when evaporation was highest, and more water was allowed to diffuse out of the devices. The thicker devices (starting with the 2 mm device) showed significantly lower warpage, with the 2.5 mm and 3 mm devices showing no warpage at all over the entire period of experimentation (30 DIV). The warpage of the devices can be attributed to hydration of the devices leading to compressive stress on the polymeric structure of the resin, and permanent warpage.

Thus, it is seen that the 3D printed polymer chemistry has a very important role to play not only is achieving optimum design-to-device translations which is dependent on the 3D printer resolution but also on biocompatibility for long term in vitro cultures. With the increasing growth in 3D printing technology, designs with significantly higher packing densities of 3D microelectrodes can be achieved along with the use of a wide variety of biocompatible polymers which can be printed in open platform 3D printers. Such a proof-of-concept device was 3D printed using the Asiga MAX X27UV (Alexandria, Australia) Digital Light Processing (DLP) 3D printer which offers a X/Y resolution of 27 μm/27 μm and a Z resolution of 1 μm. FIG. 8 (e) depicts an SEM image of a high density 3D MEA with 131 recording/stimulating sites compatible with the Nerve-On-A-Chip®[46] platform. The base diameter of the electrodes is ˜100 μm with a height of ˜150 μm. A biocompatible build material (Pro3dure GR-1 CLEAR, Protoproducts, NY, USA) was used as polymer material to print using the open platform of the 3D printer.

### CONCLUSIONS

In this work we have demonstrated the rapid fabrication of a novel 3D microtower MEAs as well as smaller, customizable electrodes having a size of 30 μm² at a height of 400 μm, with a 3D printing-based microfabrication technology and its integration with a microengineered Nerve-On-A-Chip® model. These MEAs are technically robust and fully functional for in vitro applications. The fabrication methodology involves the application of ‘Makerspace Microfabrication’ techniques, a new concept in micro/nanofabrication that demonstrates a close synergy between conventional semiconductor technologies and non-traditional, benchtop micromachining approaches. The electrical and electrochemical characteristics of the 3D MEAs show comparable performance with 3D MEAs realized using much more sophisticated, elaborate and cost intensive techniques. Biocompatibility studies demonstrated favorable cell viability on the MEAs, with the components of the device leading to a detailed chemical understanding of the base resin and its possible cytotoxic components. A stress test of the resin material established design framework for longer term assays with the MEAs. Such an integration between 3D printed, 3D MEAs and a 3D microengineered Nerve-On-A-Chip® model provides for a system ready for “disease in a dish” and “organ on a chip” applications of cell/tissue growth, proliferation and long-term cultures in-vitro toward rapid pharmaceutical, chemical and environmental screening.

### REFERENCES CITED IN THIS EXAMPLE

1. DiMasi, J. A., H. G. Grabowski, and R. W. Hansen, Innovation in the pharmaceutical industry: new estimates of R&D costs. Journal of health economics, 2016. 47: p. 20-33.
2. Kola, I. and J. Landis, Can the pharmaceutical industry reduce attrition rates? Nature reviews Drug discovery, 2004. 3(8): p. 711.
3. Skardal, A., T. Shupe, and A. Atala, Organoid-on-a-chip and body-on-a-chip systems for drug screening and disease modeling. Drug discovery today, 2016. 21(9): p. 1399-1411.
4. Zhang, B. and M. Radisic, Organ-on-a-chip devices advance to market. Lab on a Chip, 2017. 17(14): p. 2395-2420.
5. Arezzo, J. C., M. S. Litwak, and E. G. Zotova, Correlation and Dissociation of Electrophysiology and Histopathology in the Assessment of Toxic Neuropathy. Toxicologic Pathology, 2010. 39(1): p. 46-51.
6. Khoshakhlagh, P., et al., Methods for fabrication and evaluation of a 3D microengineered model of myelinated peripheral nerve. J Neural Eng, 2018. 15(6): p. 064001.
7. Sharma, A. D., et al., Human Nerve-On-A-Chip®. Engineering 3D functional human peripheral nerve in vitro. bioRxiv, 2018: p. 458463.
8. Lewandowska, M. K., et al., Recording large extracellular spikes in microchannels along many axonal sites from individual neurons. PLoS One, 2015. 10(3): p. e0118514-e0118514.
9. Radivojevic, M., et al., Tracking individual action potentials throughout mammalian axonal arbors. eLife, 2017. 6: p. e30198.
10. Rajaraman, S., et al., Microfabrication technologies for a coupled three-dimensional microelectrode, microfluidic array. Journal of Micromechanics and Microengineering, 2006. 17(1): p. 163.
11. Rajaraman, S., et al., Metal-transfer-micromolded three-dimensional microelectrode arrays for in-vitro brain-slice recordings. Journal of Microelectromechanical Systems, 2011. 20(2): p. 396-409.
12. Bhatia, S. N. and D. E. Ingber, Microfluidic organs-on-chips. Nature biotechnology, 2014. 32(8): p. 760.
13. Ruther, P., et al., The NeuroProbes project-multifunctional probe arrays for neural recording and stimulation. Biomed. Tech, 2008. 53(1): p. 238-240.
14. Kim, R., et al., Recent trends in microelectrode array technology for in vitro neural interface platform. Biomedical Engineering Letters, 2014. 4(2): p. 129-141.
15. Ko, K. R. and J. P. Frampton, Developments in 3D neural cell culture models: the future of neurotherapeutics testing?, 2016, Taylor & Francis.
16. LaPlaca, M. C., et al., Three-dimensional neuronal cultures. Methods in bioengineering: 3D tissue engineering, 2010: p. 187-204.
17. Choi, S. H., et al., A three-dimensional human neural cell culture model of Alzheimer's disease. Nature, 2014. 515(7526): p. 274.
18. Khudhair, D., et al., Microelectrode Arrays: Architecture, Challenges and Engineering Solutions, in Emerging Trends in Neuro Engineering and Neural Computation 2017, Springer. p. 41-59.
19. Fang, S.-P., et al. A carbon nanofiber (CNF) based 3-D microelectrode array for in-vitro neural proliferation and signal recording. in Micro Electro Mechanical Systems (MEMS), 2016 IEEE 29th International Conference on. 2016. IEEE.
20. Charvet, G., et al., BioMEA™: A versatile high-density 3D microelectrode array system using integrated electronics. Biosensors and Bioelectronics, 2010. 25(8): p. 1889-1896.
21. Ghane Motlagh, B., High-density 3D pyramid-shaped microelectrode arrays for brain-machine interface applications, 2015, Ecole Polytechnique de Montreal.
22. Rajaraman, S., Micromachining Techniques for Realization of Three-Dimensional Microelectrode Arrays, in Nanotechnology and Neuroscience: Nano-electronic, Photonic and Mechanical Neuronal Interfacing 2014, Springer. p. 135-182.
23. Wise, K. D., et al., Wireless implantable microsystems: high-density electronic interfaces to the nervous system. Proceedings of the IEEE, 2004. 92(1): p. 76-97.
24. Wise, K. D., Silicon microsystems for neuroscience and neural prostheses. IEEE engineering in medicine and biology magazine, 2005. 24(5): p. 22-29.
25. Merriam, S. M. E., et al., A three-dimensional 64-site folded electrode array using planar fabrication. Journal of Microelectromechanical Systems, 2011. 20(3): p. 594-600.
26. Jones, K. E., P. K. Campbell, and R. A. Normann, A glass/silicon composite intracortical electrode array. Annals of biomedical engineering, 1992. 20(4): p. 423-437.
27. Rousche, P. J. and R. A. Normann, Chronic recording capability of the Utah Intracortical Electrode Array in cat sensory cortex. Journal of neuroscience methods, 1998. 82(1): p. 1-15.
28. Bhandari, R., et al., A novel masking method for high aspect ratio penetrating microelectrode arrays. Journal of Micromechanics and Microengineering, 2009. 19(3): p. 035004.
29. Branner, A., R. B. Stein, and R. A. Normann, Selective stimulation of cat sciatic nerve using an array of varying-length microelectrodes. Journal of neurophysiology, 2001. 85(4): p. 1585-1594.
30. Kisban, S., et al. Microprobe array with low impedance electrodes and highly flexible polyimide cables for acute neural recording. in Engineering in Medicine and Biology Society, 2007. EMBS 2007. 29th Annual International Conference of the IEEE. 2007. IEEE.
31. Herwik, S., et al., Fabrication technology for silicon-based microprobe arrays used in acute and sub-chronic neural recording. Journal of Micromechanics and Microengineering, 2009. 19(7): p. 074008.
32. Aarts, A., et al. A 3D slim-base probe array for in vivo recorded neuron activity. in Engineering in Medicine and Biology Society, 2008. EMBS 2008. 30th Annual International Conference of the IEEE. 2008. IEEE.
33. Aarts, A., 3D Interconnect Technology for Out-of-Plane Biomedical Probe Arrays. A Modular Approach with Slim-Base Solutions (3D Interconnect technologie voor orthogonale biomedische microprobe arrays. Een modulaire aanpak). 2011.
34. Spieth, S., et al. Robust microprobe systems for simultaneous neural recording and drug delivery. in 4th European Conference of the International Federation for Medical and Biological Engineering. 2009. Springer.
35. Spieth, S., et al., A floating 3D silicon microprobe array for neural drug delivery compatible with electrical recording. Journal of Micromechanics and Microengineering, 2011. 21(12): p. 125001.
36. Musallam, S., et al., A floating metal microelectrode array for chronic implantation. Journal of neuroscience methods, 2007. 160(1): p. 122-127.
37. Metz, S., et al. Microelectrodes with three-dimensional structures for improved neural interfacing. in Engineering in Medicine and Biology Society, 2001. Proceedings of the 23rd Annual International Conference of the IEEE. 2001. IEEE.
38. Fofonoff, T. A., et al., Microelectrode array fabrication by electrical discharge machining and chemical etching. IEEE transactions on biomedical engineering, 2004. 51(6): p. 890-895.
39. Rousche, P. J., et al., Flexible polyimide-based intracortical electrode arrays with bioactive capability. IEEE Transactions on biomedical engineering, 2001. 48(3): p. 361-371.
40. Maher, M., et al., The neurochip: a new multielectrode device for stimulating and recording from cultured neurons. Journal of neuroscience methods, 1999. 87(1): p. 45-56.
41. Owens, A. L., et al., Multi-electrode array for measuring evoked potentials from surface of ferret primary auditory cortex. Journal of neuroscience methods, 1995. 58(1-2): p. 209-220.
42. Rodger, D. C. and Y.-C. Tai, Microelectronic packaging for retinal prostheses. IEEE Engineering in Medicine and Biology Magazine, 2005. 24(5): p. 52-57.
43. Walsh III, D. I., et al., Enabling microfluidics: from clean rooms to makerspaces. Trends in biotechnology, 2017. 35(5): p. 383-392.
44. Kundu, A., T. Ausaf, and S. Rajaraman, 3D Printing, Ink Casting and Micromachined Lamination (3D PICLμM): A Makerspace Approach to the Fabrication of Biological Microdevices. Micromachines, 2018. 9(2): p. 85.
45. Nilab, A., et al., Fabrication and characterization of 3D printed, 3D microelectrode arrays with spin coated insulation and functional electrospun 3D scaffolds for “disease in a dish” and “organ on a chip” models Hilton Head Workshop 2018: A Solid-State Sensors, Actuators and Microsystems Workshop, 2018.
46. Huval, R. M., et al., Microengineered peripheral Nerve-On-A-Chip® for preclinical physiological testing. Lab on a Chip, 2015. 15(10): p. 2221-2232.
47. Mortimer, J. T. and N. Bhadra, Peripheral nerve and muscle stimulation, in Neuroprosthetics: theory and practice 2004, World Scientific. p. 638-682.
48. Wang, R., et al., Fabrication and characterization of a parylene-based three-dimensional microelectrode array for use in retinal prosthesis. Journal of Microelectromechanical Systems, 2010. 19(2): p. 367-374.
49. Kim, J.-H., et al., Surface-modified microelectrode array with flake nanostructure for neural recording and stimulation. Nanotechnology, 2010. 21(8): p. 085303.
50. Rajaraman, S., J. D. Ross, and A. Preyer, Devices, systems and methods for high-throughput electrophysiology, 2016, Google Patents.
51. Naderi, N., M. Hashim, and J. Rouhi, Synthesis and Characterization of Pt Nanowires Electrodeposited into the Cylindrical Pores of Polycarbonate Membranes. Int. J. Electrochem.
Sci, 2012. 7: p. 8481-8486.
52. www.sigmaaldrich.com/MSDS/MSDS/DisplayMSDSPage.do?country=US&language=en&productNumber=200336&brand=ALDRICH&PageToGoToURL=https%3A%2F%2Fwww.sigmaaldrich.com %2Fcatalog%2Fproduct%2Faldrich%2F200336%3Flang%3Den
53. patents.justia.com/patent/20120125672
54. Carve, M. and D. Wlodkowic, 3D-printed chips: Compatibility of additive manufacturing photopolymeric substrata with biological applications. Micromachines, 2018. 9(2): p. 91.
55. www.radtech.org/proceedings/2012/papers/end-user-presentations/LED/DSM_Rundlett_LED.pdf
56. Bertolo, M. V. L., et al., Influence of Photoinitiator System on Physical-Chemical Properties of Experimental Self-Adhesive Composites. Brazilian dental journal, 2017. 28(1): p. 35-39.
57. patents.justia.com/patent/20120125672
58. www.sigmaaldrich.com/MSDS/MSDS/DisplayMSDSPage.do?country=US&language=en&productNumber=252379&brand=SIGALD&PageToGoToURL=https%3A%2F%2Fwww.sigmaaldrich.com%2Fcatalog%2Fproduct%2Fsigald%2F252379%3Flang%3Den
59. www.sigmaaldrich.com/MSDS/MSDS/DisplayMSDSPage.do?country=US&language=en&productNumber=415952&brand=ALDRICH&PageToGoToURL=https %3A %2F %2Fwww.sigmaaldrich.com%2Fcatalog%2Fproduct%2Faldrich%2F415952%3Flang%3Den

### Example 4

TABLE S1
Comparative Table for In-Vitro 3 D MEAs

| Reference | Material | Method | Remarks |
|---|---|---|---|
| 1 | Silicon and SU-8 | This structure is created by | Involves use of clean room |
| | | fabricating, aligning, and stacking | and multiple photolithography |
| | | individually patterned thin films, each | steps. |
| | | of which constitutes an electrode to | |
| | | achieve 3 dimensionality. | |
| 2 | (a) Flexible, | (a) Mold of anisotropically etched | Involves use of clean room |
| | implantable | silicon for fabricating the flexible 3 D | and wet etching to achieve |
| | microelectrodes with | polymide MEA. | 3 D MEAs. |
| | pyramidal, | (b) Wet chemical etching of glass to | |
| | protruding structures | achieve 3 D glass electrodes. | |
| | using Silicon and | | |
| | Polymide and (b) tip- | | |
| | shaped electrode | | |
| | arrays on glass | | |
| | substrates | | |
| 3 | Titanium, titanium- | Electrical Discharge Machining and | The machining process is |
| | aluminum-vanadium | Chemical Etching. | elaborate and the spacing |
| | alloy (Ti90—Al6—V4), | | between electrodes is limited |
| | stainless steels, and | | by the size of the wire used in |
| | tungsten carbide. | | the EDM process. |
| 4 | Polydimethylsiloxane | Process consists of (a) designing and | Multiple steps required to |
| | (PDMS) substrate, | etching the stretchable MEA's | fabricate the final device |
| | conductive-PDMS | microneedles, (b) producing the | involving the use of Silicon- |
| | traces, and stainless- | negative micromold for the device, | wafers which were patterned |
| | steel penetrating | (c) micromolding the stretchable | via inductively coupledplasma |
| | electrodes. | MEA, and (d) packaging the device. | (ICP) etching technology to |
| | | | produce negative molds, |
| | | | with which to form the |
| | | | conductive traces of the |
| | | | stretchable MEAs. |
| 5 | Metal microneedles | Metal transfer micromolding is | Involves use of clean room |
| | on SU-8, PMMA, | introduced as a manufacturing | and multiple photolithography |
| | and PU. | technology for 3 D MEAs on | steps. |
| | | polymeric substrates. | |
| 6 | SU-8 towers on fused | Fabrication of the towers included | Involves use of clean room |
| | silica substrates. | double-sided exposure technology to | and multiple photolithography |
| | | create high aspect ratio structures in | steps and an assembly of the |
| | | SU-8. | high aspect ratio SU-8 |
| | | | structures on fused silica |
| | | | substrate. |
| 7 | (PDMS)-based | 3 D MEAs fabricated with surface- | Involves use of clean room |
| | MEAs featuring | mounting structure by the molding | and multiple fabrication steps |
| | plateau-shaped | technique. PDMS-based MEA do not | involving lithography and |
| | microelectrodes. | generate a recessed structure, and | Reactive Ion Etching to |
| | | instead produces a plateau-shaped | fabricate the final device. |
| | | electrode. | |
| 8 | Metal electrodes on | The electrode has a multiple | Involves usage of |
| | flexible polyimide | metallization layer architecture in | microfabrication and thin-film |
| | substrate | which the routing tracks are layered | processing. |
| | | underneath the large area pads. The | |
| | | multishank planar electrode is used | |
| | | for creating the 3-D “Waterloo Array” | |
| | | using custom designed stackers. | |
| 9 | Low-temperature co- | Facile realization of 3 D hybrid | Assembling of multiple |
| | fired ceramics | devices based on complex | components required. |
| | (LTCC) for the | multilayer assemblies. | |
| | design of a 3- | | |
| | dimensional | | |
| | multi-electrode array | | |
| | (3 D MEA). | | |
| 10 | 3 D silicon pillars | Combination of chemical | Involves usage of |
| | with SiNx insulation, | polymerization methods and micro- | microfabrication and thin-film |
| | | fabrication techniques for creating | processing in a clean room |
| | | conducting polymer pillar electrodes | environment. |
| 11 | Gold mushroom- | Photolithography for definition of | Involves use of lithographic |
| | shaped | electrodes and electroplating for | techniques in a clean room |
| | micro-protrusion | achieving 3 Dimensionality. | environment. |
| | matrices | | |
| | were prepared glass | | |
| | wafer | | |

### REFERENCES CITED IN THIS EXAMPLE

1. Musick, K.; Khatami, D.; Wheeler, B. C., Three-dimensional micro-electrode array for recording dissociated neuronal cultures. Lab Chip 2009, 9 (14), 2036-2042.
2. Metz, S.; Heuschkel, M. O.; Avila, B. V.; Holzer, R.; Bertrand, D.; Renaud, P. In Microelectrodes with three-dimensional structures for improved neural interfacing, Engineering in Medicine and Biology Society, 2001. Proceedings of the 23rd Annual International Conference of the IEEE, IEEE: 2001; pp 765-768.
3. Fofonoff, T. A.; Martel, S. M.; Hatsopoulos, N. G.; Donoghue, J. P.; Hunter, I. W., Microelectrode array fabrication by electrical discharge machining and chemical etching. IEEE transactions on biomedical engineering 2004, 51 (6), 890-895.
4. Guvanasen, G. S.; Guo, L.; Aguilar, R. J.; Cheek, A. L.; Shafor, C. S.; Rajaraman, S.; Nichols, T. R.; DeWeerth, S. P., A Stretchable Microneedle Electrode Array for Stimulating and Measuring Intramuscular Electromyographic Activity. IEEE Transactions on Neural Systems and Rehabilitation Engineering 2017, 25 (9), 1440-1452.
5. Rajaraman, S.; Choi, S.-O.; McClain, M. A.; Ross, J. D.; LaPlaca, M. C.; Allen, M. G., Metal-transfer-micromolded three-dimensional microelectrode arrays for in-vitro brain-slice recordings. Journal of Microelectromechanical Systems 2011, 20 (2), 396-409.
6. Rajaraman, S.; Choi, S.-O.; Shafer, R. H.; Ross, J. D.; Vukasinovic, J.; Choi, Y.; DeWeerth, S. P.; Glezer, A.; Allen, M. G., Microfabrication technologies for a coupled three-dimensional microelectrode, microfluidic array. J. Micromech. Microeng 2006, 17 (1), 163.
7. Kim, J.-M.; Im, C.; Lee, W. R., Plateau-Shaped Flexible Polymer Microelectrode Array for Neural Recording. Polymers 2017, 9 (12), 690.
8. Gabran, S.; Salam, M. T.; Dian, J.; El-Hayek, Y.; Velazquez, J. P.; Genov, R.; Carlen, P. L.; Salama, M.; Mansour, R. R., 3-D flexible nano-textured high-density microelectrode arrays for high-performance neuro-monitoring and neuro-stimulation. IEEE Transactions on Neural Systems and Rehabilitation Engineering 2014, 22 (5), 1072-1082.
9. Tech, J. C. S., Ltcc-based multi-electrode arrays for 3d in vitro cell cultures. 2015.
10. Sasso, L.; Vazquez, P.; Vedarethinam, I.; Castillo-Leon, J.; Emnéus, J.; Svendsen, W. E., Conducting polymer 3D microelectrodes. Sensors 2010, 10 (12), 10986-11000.
11. Ojovan, S. M.; Rabieh, N.; Shmoel, N.; Erez, H.; Maydan, E.; Cohen, A.; Spira, M. E., A feasibility study of multi-site, intracellular recordings from mammalian neurons by extracellular gold mushroom-shaped microelectrodes. Scientific reports 2015, 5, 14100.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

We claim:

1. A three-dimensional microelectrode array comprising:
a chip that further comprises at least one two-dimensional electrode, at least one three-dimensional electrode, or a combination thereof;
wherein the microelectrode array is configured to provide real-time, reliable detection of one or more bioelectrical signals in a microengineered physiological system.

2. The microelectrode array of claim 1, wherein the one or more bioelectrical signals comprise single action potentials, compound action potentials, high frequency waves, low frequency waves, or a combination thereof.

3. The microelectrode array of claim 1, wherein the microengineered physiological system comprises a tissue explant, a suspension of cells, or a combination thereof.

4. The microelectrode array of claim 1, wherein:
the microengineered physiological system comprises neural cells cultured on a micropatterned platform or tissue explants seeded on a micropatterned platform, wherein the micropatterned platform permits the formation of a neural architecture; and
the microelectrode array comprises an area with a configuration that is complementary to that of the neural architecture.

5. The microelectrode array of claim 4, wherein the neural architecture comprises an axonal growth region, a ganglion region, a dendritic region, a synaptic region, a spheroid region, or a combination thereof.

6. The microelectrode array of claim 5, comprising a first plurality of electrodes positioned in the ganglion region or spheroid region and a second plurality of electrodes positioned at defined intervals down the axonal growth region.

7. The microelectrode array of claim 6, wherein the first plurality of electrodes, the second plurality of electrodes, or both comprise recording electrodes, stimulation electrodes, or a combination thereof.

8. The microelectrode array of claim 6, wherein the first plurality of electrodes comprises at least one planar electrode, the second plurality of electrodes comprises at least one three-dimensional electrode, or vice versa.

9. The microelectrode array of claim 6, wherein the defined intervals comprise up to about 5 mm intervals.

10. The microelectrode array of claim 6, wherein the microelectrode array comprises up to about sixty-four electrodes.

11. The microelectrode array of claim 6, wherein the first plurality of electrodes comprises up to ten electrodes, the second plurality of electrodes comprises up to ten electrodes, or a combination thereof.

12. The microelectrode array of claim 4, configured to accommodate at least 16 three-dimensional electrodes, at least 16 planar electrodes, or a combination thereof within the area that is complementary to that of the neural architecture.

13. The microelectrode array of claim 1, wherein the microelectrode array is configured to detect one or more bioelectric signals of at least 10 μV.

14. The microelectrode array of claim 1, wherein the microelectrode array is configured to detect one or more bioelectric signals in a microengineered physiological system for up to one year.

15. The microelectrode array of claim 14, wherein the microelectrode array is configured to detect one or more bioelectric signals in a microengineered physiological system for up to about eight weeks.

16. The microelectrode array of claim 1, wherein the microelectrode array comprises a biocompatible conductive ink, a biocompatible conductive paste, a biocompatible conductive composite, or a combination thereof.

17. The microelectrode array of claim 1, wherein the microelectrode array further comprises one or more vias.

18. The microelectrode array of claim 1, further comprising an insulation layer.

19. The microelectrode array of claim 18, wherein the insulation layer comprises a material that is biocompatible.

20. The microelectrode array of claim 18, wherein the insulation layer comprises parylene, poly-di-methyl-siloxane (PDMS), SU-8, silicon dioxide, polyimide, polyurethane, poly lactic acid, poly glycolic acid, poly lactic glycolic acid, poly vinyl alcohol, polystyrene, poly ethylene glycol, poly ethylene terephthalate, poly ethylene terephthalate glycol, poly ethylene naphthalate, or a combination thereof.

21. The microelectrode array of claim 1, further comprising volumetric stimulators configured to stimulate the microengineered physiological system.

22. The microelectrode array of claim 1, wherein the electrodes comprise a diameter of about 50 μm or less.

23. The microelectrode array of claim 1, wherein the electrodes comprise a diameter of about up to about 1000 μm.

24. The microelectrode array of claim 1, wherein the electrodes comprise a diameter of about 30 μm or less.

25. The microelectrode array of claim 1, wherein the electrodes comprise a diameter of about 30-50 μm.

26. The microelectrode array of claim 1, wherein the at least one three-dimensional electrode comprises a tip that further comprises a radius of curvature (ROC) that is between 1 μm and 1 mm, inclusive.

27. The microelectrode array of claim 1, wherein the at least one three-dimensional electrode comprises a tip that further comprises a radius of curvature (ROC) of about 15 μm.

28. The microelectrode array of claim 1, wherein the microelectrode array is comprised of a biocompatible material.

29. The microelectrode array of claim 28, wherein the microelectrode arrays are configured to maintain viability of neuronal cells.

30. The microelectrode array of claim 1, wherein the microengineered physiological system comprises at least one neuronal cell with a structure analogous to peripheral nerve anatomy.

31. The microelectrode array of claim 1, wherein the three-dimensional microelectrodes comprise microneedle-type electrodes.

32. The microelectrode array of claim 1, wherein the at least one three-dimensional electrode comprises a height up to about 1000 μm.

33. The microelectrode array of claim 1, wherein the at least one three-dimensional electrode comprises a height of between about 300 μm to about 1000 μm.

34. The microelectrode array of claim 1, wherein the at least one three-dimensional electrode comprises a height of up to about 150 μm.

35. The microelectrode array of claim 1, wherein the at least one three-dimensional electrode comprises a height of between about 50 μm to about 150 μm.

36. The microelectrode array of claim 1, wherein the chip is configured to interface with standard commercial multichannel systems and standard commercial recording amplifiers.

37. The microelectrode array of claim 1, wherein the microelectrode array is configured to measure compound action potentials for an inference of conduction velocity, amplitude, integral, excitability after compound administration, threshold, sensitivity, CAP time width, CAP waveform shape, or a combination thereof.

38. The microelectrode array of claim 1, wherein the microelectrode array comprises a conductive trace layer.

39. The microelectrode array of claim 38, wherein the conductive trace layer comprises titanium, titanium nitride, iridium oxide, platinum, gold, aluminum, stainless steel, indium tin oxide, or a combination thereof.

40. The microelectrode array of claim 38, wherein the conductive trace layer comprises a conductive polymer.

41. The microelectrode array of claim 1, wherein the microelectrode array comprises a conductive trace layer, a polyethylene terephthalate insulation layer, micro-towers, or a combination thereof.

42. The microelectrode array of claim 41, wherein the at least one micro-tower is coated with micro-porous platinum, nano-porous platinum, nano-gold, or a combination thereof.

43. The microelectrode array of claim 1, wherein the microelectrode array comprises a titanium/gold metal trace.

44. The microelectrode array of claim 1, wherein the microelectrode array comprises a titanium/aluminum trace layer and a silicon dioxide insulation layer.

45. A system for reproducibly detecting compound action potentials in microengineered physiological system, the system comprising a microelectrode array; and
a microphysiological system comprising one or more neuronal cells;
wherein the microelectrode array comprises the microelectrode array of claim 1.

46. The system of claim 45, wherein the microengineered physiological system is grown upon or transferred to the microelectrode array.

47. The system of claim 45, wherein the one or more neural cells comprise peripheral nervous system neurons, central nervous system neurons, Schwann cells, oligodendrocytes, microglial cells, glial cells, other peripheral or central nervous support cells, or a combination thereof.

48. The system of claim 45, wherein the one or more neuronal cells comprise sensory neurons, interneurons, or motor neurons.

49. The system of claim 47, wherein the peripheral nervous system neurons comprise at least one dorsal root ganglion neuron.

50. A method of predicting the type and severity a neural pathology comprising:
growing sample neural tissue on or transferring neural tissue to the microelectrode array of claim 1, wherein the sample neural tissue comprises an axonal growth region and a ganglion region;
electrophysiological testing to determine the nerve conduction velocity of the sample neural tissue, wherein electrophysiological testing comprises electrically stimulating at least one location along the axonal growth region, the ganglion region, or a combination thereof and recording from at least one location within the ganglion region, the axonal growth region, or a combination thereof; and
comparing nerve conduction velocity obtained from sample neural tissue to that of neural tissue that is known to be healthy neural tissue;
wherein reduced nerve conduction in the sample neural tissue as compared to the healthy neural tissue indicates a neural pathology.

51. The method of claim 50, further comprising histological analysis of the neural tissue.

52. The method of claim 51, wherein histological analysis comprises an assessment of axon diameter, axon density, myelination, cell morphology, cell type, nerve structure, or a combination thereof.

53. The method of claim 50, wherein the electrophysiological testing further comprises stimulating a plurality of locations along the axonal growth region, the ganglion region, or a combination thereof and recording a resultant electrical response from the ganglion region, the axonal growth region, or a combination thereof.

54. The method of claim 50, wherein the electrophysiological testing is performed over a multi-week period to chronically measure neurodegeneration.

55. A method of assessing a response from neural tissue comprising:
growing neural tissue upon or transferring to the microelectrode array of claim 1;
introducing one or more stimuli to the neural tissue; and
measuring one or more responses from the neural tissue to the one or more stimuli,
wherein the one or more responses comprise compound action potential amplitude, conduction velocity, waveform shape, histomorphological parameters, or combination thereof.

56. The method of claim 55, wherein introducing the one or more stimuli comprises contacting the neural tissue with at least one pharmacologically active compound, electrical stimulus, chemical stimulus, optical stimuli, physical stimuli, or a combination thereof.

57. A method of evaluating the toxicity of an agent comprising:
growing neural tissue on or transferring neural tissue to the microelectrode array of claim 1;
exposing at least one agent to the neural tissue;
measuring or observing changes in compound action potential amplitude, conduction velocity, waveform shape, histomorphological parameters, or combination thereof; and
correlating any measured or observed changes of the neural tissue with the toxicity of the agent, such that,
if the measured or observed changes are indicative of decreased cell viability, the agent is characterized as toxic and,
if the measured or observed changes are indicative of unchanged or increased cell viability, the agent is characterized as non-toxic.

58. A method of measuring myelination or demyelination of one or more axons of one or a plurality of neuronal cells, comprising:
growing neural tissue on or transferring neural tissue to the microelectrode array of claim 1 under conditions sufficient to grow at least one axon;
inducing a compound action potential in the neural tissue;
measuring the compound action potential; and
quantifying the levels of myelination of the neural tissue based on the compound action potential.

59. A method of fabricating a three-dimensional microelectrode array comprising:
processing a chip to accommodate a plurality of electrodes, a plurality of vias, or a combination thereof;
metallization of the plurality of electrodes using a shadow mask;
screen printing of conductive inks;
curing the conductive ink in an oven;
depositing insulation onto the conductive ink and metalized electrodes;
defining the recording sites of the plurality of electrodes; and
combining a printed circuit board with the chip.

60. The method of claim Error! Reference source not found., wherein insulation is deposited over the entirety of the processing chip.

61. The method of claim Error! Reference source not found., further comprising fabricating conductive vias for top to bottom signal transduction.
